(19) United States
(12) Patent Application Publication
(10) Pub. No.: US 2024/0411370 A1
(43) Pub. Date: Dec. 12, 2024

(54) FITNESS DEVICE GAMING CONTROLLER

(71) Applicant: Blue Goji LLC, Austin, TX (US)

(72) Inventor: Coleman Fung, Austin, TX (US)

(21) Appl. No.: 18/744,625

(22) Filed: Jun. 15, 2024

Related U.S. Application Data
(63) Continuation-in-part of application No. 18/434,561, filed on Feb. 6, 2024.
Application No. 18/434,561 is a continuation-in-part of application No. 18/433,350, filed on Feb. 5, 2024.
Application No. 18/433,350 is a continuation-in-part of application No. 18/299,017, filed on Apr. 11, 2023.
Application No. 18/299,017 is a continuation-in-part of application No. 18/171,330, filed on Feb. 18, 2023, now Pat. No. 11,914,776.
Application No. 18/171,330 is a continuation of application No. 17/030,233, filed on Sep. 23, 2020, now Pat. No. 11,662,818.
Application No. 17/030,233 is a continuation-in-part of application No. 17/030,195, filed on Sep. 23, 2020.
Application No. 17/030,195 is a continuation-in-part of application No. 16/781,663, filed on Feb. 4, 2020, now Pat. No. 11,191,996.
Application No. 16/781,663 is a continuation-in-part of application No. 16/354,374, filed on Mar. 15, 2019, now Pat. No. 10,549,153.
Application No. 16/354,374 is a continuation-in-part of application No. 16/176,511, filed on Oct. 31, 2018, now Pat. No. 10,960,264.
Application No. 16/176,511 is a continuation-in-part of application No. 16/011,394, filed on Jun. 18, 2018, now Pat. No. 10,155,133.
Application No. 16/011,394 is a continuation-in-part of application No. 15/853,746, filed on Dec. 23, 2017, now Pat. No. 10,265,578.
Application No. 15/853,746 is a continuation of application No. 15/219,115, filed on Jul. 25, 2016, now Pat. No. 9,849,333.
Application No. 15/219,115 is a continuation-in-part of application No. 15/193,112, filed on Jun. 27, 2016, now abandoned.
Application No. 15/193,112 is a continuation-in-part of application No. 15/187,787, filed on Jun. 21, 2016, now Pat. No. 10,124,255.
Application No. 15/187,787 is a continuation-in-part of application No. 15/175,043, filed on Jun. 7, 2016, now Pat. No. 9,766,696.
Application No. 15/187,787 is a continuation-in-part of application No. 14/846,966, filed on Sep. 7, 2015, now Pat. No. 10,080,958.
Application No. 14/846,966 is a continuation-in-part of application No. 14/012,879, filed on Aug. 28, 2013, now Pat. No. 10,737,175.
Application No. 17/030,233 is a continuation-in-part of application No. 16/927,704, filed on Jul. 13, 2020, now abandoned.
Application No. 16/927,704 is a continuation of application No. 16/867,238, filed on May 5, 2020, now abandoned.
Application No. 16/867,238 is a continuation-in-part of application No. 16/793,915, filed on Feb. 18, 2020, now abandoned.
Application No. 16/793,915 is a continuation-in-part of application No. 16/255,641, filed on Jan. 23, 2019, now Pat. No. 10,561,900.
Application No. 16/255,641 is a continuation of application No. 16/223,034, filed on Jan. 23, 2019, now Pat. No. 10,688,341.
Application No. 16/223,034 is a continuation-in-part of application No. 16/176,511, filed on Oct. 31, 2018, now Pat. No. 10,960,264.
Application No. 18/434,561 is a continuation of application No. 18/515,072, filed on Nov. 20, 2023, now abandoned.
Application No. 18/515,072 is a continuation of application No. 18/463,270, filed on Sep. 7, 2023, now abandoned.
Application No. 18/463,270 is a continuation of application No. 18/335,070, filed on Jun. 14, 2023, now abandoned.
Application No. 18/335,070 is a continuation of application No. 16/990,728, filed on Aug. 11, 2020, now Pat. No. 11,756,664.
Application No. 16/990,728 is a continuation of application No. 14/012,879, filed on Aug. 28, 2013, now Pat. No. 10,737,175.
(60) Provisional application No. 62/310,568, filed on Mar. 18, 2016.
(60) Provisional application No. 62/330,602, filed on May 2, 2016.
(60) Provisional application No. 62/330,642, filed on May 2, 2016.
(60) Provisional application No. 62/697,973, filed on Jul. 13, 2018.
(60) Provisional application No. 61/696,068, filed on Aug. 31, 2012.

(51) Int. Cl. G06F 3/01; A61B 5/00; A61B 5/11; A61B 5/16; A63B 22/00; A63B 22/02; A63B 22/06; A63B 23/04; A63B 24/00; A63F 13/212; A63F 13/214; A63F 13/40; A63F 13/65; G06F 1/16; G06F 3/03; G06T 19/00; H04W 84/18

(52) U.S. Cl. CPC G06F 3/015; A61B 5/165; A61B 5/4082; A61B 5/4088; A61B 5/4842; A63B 22/0046; A63B 22/0285; A63B 22/0292; A63B 22/06; A63B 23/04; A63F 13/212; A63F 13/214; A63F 13/40; A63F 13/65; G06F 1/163; G06F 3/011; G06F 3/016; G06T 19/006; A61B 5/1124; A63B 24/0003; G06F 3/0304; G06F 2203/012; H04W 84/18

(57) ABSTRACT

A fitness device gaming controller to be used to control an application running on a host device. The fitness device operates as a normal piece of exercise equipment, with the capability of turning a user's monitored exercise activity and manual controls into standard gaming inputs for use by a gaming device such as a console or computer. Additional devices may be connected to the fitness device, such as additional sensors or smart devices that can provide additional sensors and controls for incorporation into the gaming inputs.

### CROSS-REFERENCE TO RELATED APPLICATIONS

Priority is claimed in the application data sheet to the following patents or patent applications, each of which is expressly incorporated herein by reference in its entirety:
Ser. No. 18/434,561
Ser. No. 18/433,350
Ser. No. 18/299,017
Ser. No. 17/405,347
Ser. No. 15/609,910
62/358,517
Ser. No. 18/171,330
Ser. No. 17/030,233
Ser. No. 17/030,195
Ser. No. 16/781,663
Ser. No. 16/354,374
Ser. No. 16/176,511
Ser. No. 16/011,394
Ser. No. 15/853,746
Ser. No. 15/219,115
Ser. No. 15/193,112
Ser. No. 15/187,787
Ser. No. 15/175,043
62/310,568
Ser. No. 14/896,966
Ser. No. 14/012,879
61/696,068
62/330,602
62/330,642
Ser. No. 16/927,704
Ser. No. 16/867,238
Ser. No. 16/793,915
Ser. No. 16/255,641
Ser. No. 16/223,034
62/697,973
Ser. No. 18/515,072
Ser. No. 18/463,270
Ser. No. 18/335,070
Ser. No. 16/990,728

### BACKGROUND OF THE INVENTION

### Field of the Invention

The disclosure generally relates to the field of electronic gaming, and in particular to using a fitness device as an electronic controller for a game.

### Discussion of the State of the Art

Gaming devices, including home consoles, personal computers, and cloud-based services, rely on standardized input from known hardware controllers such as keyboard/mouse or handheld gaming controllers as used by consoles such as PLAYSTATION™ or XBOX™ devices. However, there is a growing trend to merge gameplay with fitness activities, often utilizing proprietary controllers to turn a fitness activity into an input the gaming device can understand, such as those seen used by NINTENDO™ WII FIT™ games which utilize gaming controllers modified to emulate the shape or function of actual fitness devices such as tennis rackets or balance boards. This forces users to purchase proprietary equipment that is only useful within the context of the associate game, and generally lacks any real fitness device function for standalone exercise or use in other activities.

What is needed, is a fitness device that operates as a gaming controller, turning a user's exercise activity and manual control inputs into standardized game input values that are expected by a gaming device, so that the user can use their fitness device across a wide variety of games as well as in normal non-gaming exercise activities.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 is a block diagram illustrating components of the adaptable fitness system, according to an aspect of the invention.

FIG. 2A is a block diagram illustrating the components of the motion sensing device, according to an aspect of the invention.

FIG. 2B illustrates a motion sensing device, according to an aspect of the invention.

FIG. 2C illustrates a hand controller, according to an aspect of the invention.

FIG. 2D illustrates an example of hand controllers mounted to handlebars of a fitness device, according to an aspect of the invention.

FIG. 3 is a flowchart illustrating an example process for interacting with a fitness application based on the user's interaction with the fitness system, according to an aspect of the invention.

FIG. 4 illustrates an example process of a user manipulating events in a fitness application based on the user's interaction with the fitness system in real time, according to an aspect of the invention.

FIG. 5 is a flow chart illustrating an example process for determining a step taken by the user, based on motion data, according to an aspect of the invention.

FIG. 6 is a flow chart illustrating an example process for interacting with the adaptable fitness system, according to an aspect of the invention.

FIG. 7 illustrates an exemplary computing environment on which an embodiment described herein may be implemented, in full or in part.

FIG. 8 is a block diagram of an exemplary system architecture, illustrating the use of nested-communication control devices with a variety of electronic systems, according to an aspect of the invention.

FIG. 9 is a flow diagram illustrating an exemplary method for natural body interaction for mixed or virtual reality applications, according to an aspect of the invention.

FIG. 10 is a block diagram of an exemplary system architecture for an exercise machine being connected over local connections to a smartphone, an output device other than a phone, and a server over a network, according to an aspect of the invention.

FIG. 11 is a flow diagram illustrating an exemplary method for processing natural body interaction and additional inputs and producing a composite output, according to an aspect of the invention.

FIGS. 12A & 12B (PRIOR ART) show a representation of a standard dual joystick controller.

FIG. 13 (PRIOR ART) shows the design and electrical characteristics of a typical thumb joystick as found in a standard dual joystick controller.

FIG. 14 shows an exemplary embodiment and the relationship between the motion sensors, standard dual joystick controllers, and the resulting image that would be seen when using an aspect of the invention to control a computer game.

FIG. 15 is a diagram showing an exemplary overall system architecture for a brainwave entrainment system using virtual objects and environments as visual, stimulation transducers.

FIG. 16 is a side view of an exemplary variable-resistance exercise machine with wireless communication for smart device control and interactive software applications, according to an aspect of the invention.

FIG. 17 is a diagram of an exemplary virtual reality or mixed reality enhanced exercise machine, illustrating the use of a stationary bicycle with hand controls on the handles, and a belt-like harness attachment.

FIG. 18 is a diagram of another exemplary virtual reality or mixed reality enhanced exercise

machine, illustrating the use of a treadmill exercise machine with a vest-type harness with a plurality of pistons to provide a hardware-based torso joystick with full-body tracking.

FIG. 19 is a diagram of another exemplary virtual reality or mixed reality enhanced exercise machine, illustrating the use of a stationary bicycle with a vest-type harness with a plurality of strain sensors and tethers.

FIG. 20 is a flow diagram illustrating an exemplary method for operating a virtual and mixed-reality enhanced exercise machine.

FIG. 21 is a diagram of another exemplary virtual reality or mixed reality enhanced exercise machine, illustrating the use of a rotating platform, a waist belt and joints providing full range of motion.

### DETAILED DESCRIPTION OF THE INVENTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

An example mobile and adaptable fitness system allows a fitness device to be used to control an application running on a host device. The system includes one or more controllers, a motion sensing device, and a host device. The hand controllers may be mounted on the handlebars of the fitness device or held in a user's hands and include buttons that the user can press. The motion sensing device may be mounted on the user or on a moving part of the fitness device and contains sensors that capture motion performed by the user. The host device receives input data from the hand controllers and the motion sensing device and controls an object in an application, such as a character, based on the input data.

A mobile and adaptable fitness system provides a significantly enhanced fitness experience for users of standard gym and fitness equipment. This adaptable fitness system can be easily connected to and removed from standard and popular fitness devices such as a step machine, elliptical machine, treadmill and the like. The system turns a fitness device into a video game controller that can be used in an application connecting to an online service. Users of the adaptable fitness system can play games and track fitness performance on a mobile host device such as tablet or smartphone.

### Overview of Adaptable Fitness System

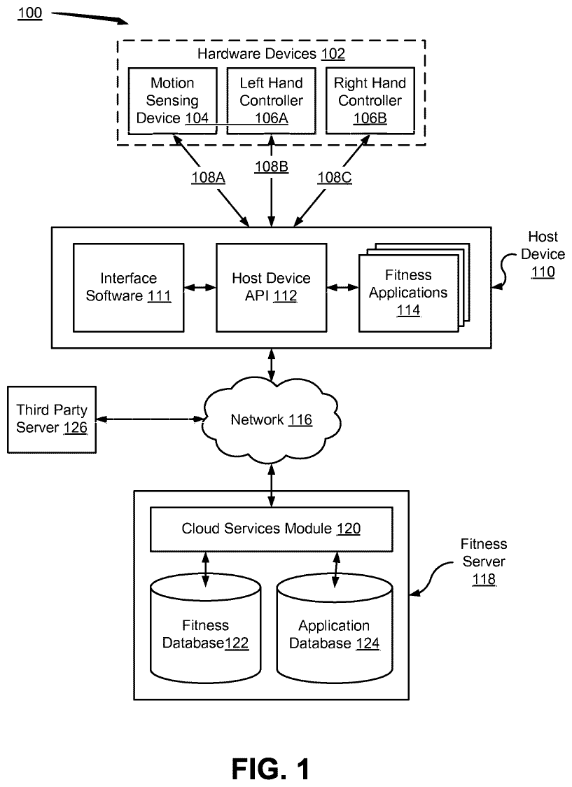

FIG. 1 is a block diagram illustrating components of the adaptable fitness system 100, according to one embodiment. The adaptable fitness system 100 includes a set of hardware devices 102, a host device 110, and a fitness server 118. In one embodiment, the hardware devices 102 generate a plurality of communication links 108A, 108B and 108C based on a user's interaction with the hardware devices 102. The host device 110 receives a plurality of data over the communication links 108A, 108B and 108C from the hardware devices 102 and interacts with a fitness application 114 or a plurality of fitness applications 114 based on the host devices' 110 interpretation of the data. The host device 110 communicates with the fitness server 118 over a network 116.

In one embodiment, the hardware devices 102 include a motion sensing device 104, a left-hand controller 106A and a right hand controller 106B. The motion sensing device 104 captures a plurality of motion data in real-time, as the user interacts with the fitness system 100. The left-hand controller 106A receives input from the user by way of an action, such as the user pressing a button on the controller or moving a joystick on the controller. The right-hand controller 106B receives input from the user by way of an action, such as the user pressing a button on the controller or moving a joystick on the controller.

In alternative embodiments, additional hardware devices 102 may be used. For example, the hardware devices 102 may also include athletic shoes with embedded sensors or motion sensors that attach directly to the fitness system. In addition, some of the hardware devices shown 102 shown in FIG. 1 may be omitted. For example, only one of the hand controllers may be used, or the two hand controllers 106A, 106B may be replaced with a combined hand controller that contains the all of the buttons that would normally be distributed between the two hand controllers 106A, 106B.

In some embodiments, multiple sets of hardware devices 102 may be added to the system 100 to adapt additional fitness devices into additional game controllers. For example, multiple stationary bicycles may be adapted into controllers for a bicycle racing application, thus allowing multiple users to participate in virtual races against each other.

As a whole, the hardware devices 102 can be used to adapt a fitness device (e.g., a step machine, an elliptical machine, a treadmill, a rowing machine, etc.) into a game controller that receives a user's physical input (both motions and button presses) and generates corresponding input data for interacting with a fitness application 114. For example, the input data could be used to control a virtual character or object in the fitness application 114. Input data from the hardware devices 102 are sent over communication links 108A, 108B, 108C (hereinafter referred to collectively as 108) to the host device API 112 on the host device 110. The communication links 108 may be wired or wireless connections.

In one embodiment, the communications links 108 are based on the Bluetooth Low Energy (BLE) protocol, and the hardware devices 102 are BLE devices. If additional sets of hardware devices 102 are included in the system, additional communication links 108 are established to connect the hardware devices 102 to the host device 110.

In the illustrated embodiment, the communication links 108 connect each hardware device 102 directly to the host device 110. In this embodiment, the three hardware devices 102 connect simultaneously to the host device 110 over the communication links 108A, 108B, 108C. The hardware devices 102 may all connect over the same communication protocol (e.g., Bluetooth Low Energy), or over different communication protocols.

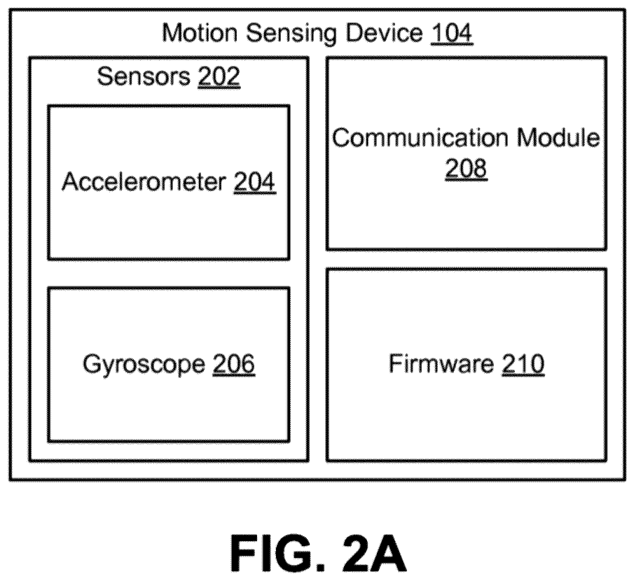

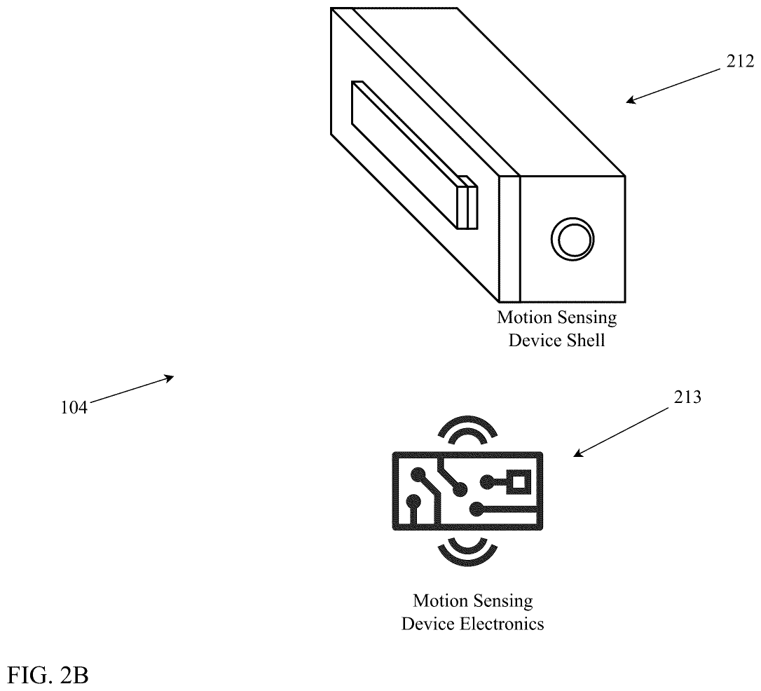

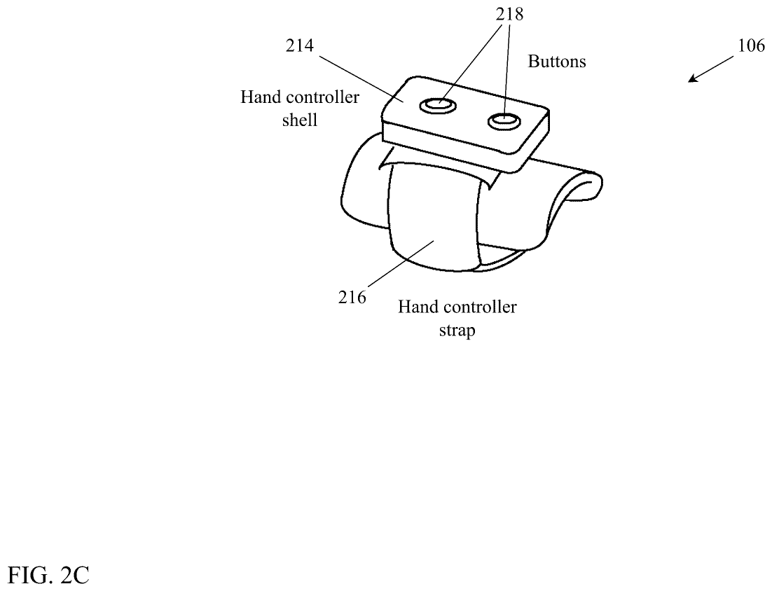

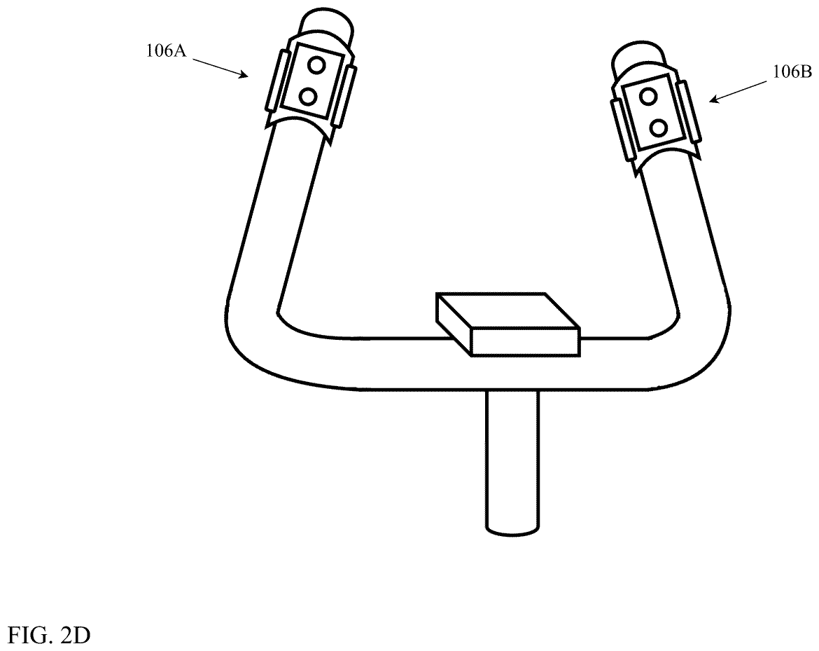

In another embodiment, only the motion sensing device 104 is connected directly to the host device 110 via the first communication link 108A. In this embodiment, the two hand controllers 106A, 106B are coupled to the motion sensing device 104 via the other two communication links 108B, 108C, and the motion sensing device 104 is configured to relay input data from the hand controllers 106A, 106B to the host device 110. This embodiment is described in more detail with reference to FIG. 2A.

The host device 110 includes interface software 111, a host device API 112, and one or more fitness applications 114. As a whole, the host device 110 is a computing device that is capable of executing program instructions. The host device 110 may be, for example, a smartphone, tablet computer, laptop computer, or desktop computer.

The interface software 111 and the host device API 112 operates in conjunction with the interface software to act as an interface between the hardware devices 102, the fitness applications 114, and the fitness server 118. When no hardware devices 102 are connected, the host device API 112 initiates a scan for hardware devices 102 at fixed intervals. Button presses on the hand controllers 106A, 106B or motion on the motion sensing device 104 will trigger the respective device to broadcast for connection (if not already connected). If the device's broadcast overlaps with one of the host device API's scanning intervals, then the host device API 112 initiates a connection attempt to the respective device. This process of establishing a connection after detecting a button press or motion allows for intuitive connection of hardware devices 102 to the host device 110. The host device API 112 maintains the connection between a fitness application 114 and a hardware device 102 as long as the application 114 remains active. After establishing a connection with one or more hardware devices 102, the interface software 111 and host device API 112 receive and process input data from the hardware devices 102 so that the input data can be used to control an application 114 or an object in an application 114. For example, the input data can be used by the application 114 to control a virtual character or object in a game.

The host device API 112 can distinguish the input data from the different hardware devices because the firmware of each hardware device can be configured to include an identifier (e.g., a binary value) in a header of any input data that is sent to the host device 110. Thus, the motion sensing device 106 would have a different identifier than the hand controllers 106A, 106B. The interface software 111 can then build a software state representation of each device based on the device's identifier, and the host device API 112 can map input data to its respective software state representation to identify the originating hardware device. After the input data is mapped, e.g., via a table or index, to the corresponding hardware device, the data can be used to as inputs to fitness applications 114. In embodiments where the motion sensing device 104 is configured to relay input data from the hand controllers 106A, 106B to the host device 110, the motion sensing device 104 may be configured to read the identifier in the input streams from the two hand controllers 106A, 106B and relay a single integrated input stream to the host device 110. This process is described in more detail with reference to FIGS. 2A and 2D.

In some embodiments, the left-and right-hand controllers 106A, 106B are manufactured to be identical (e.g., to reduce manufacturing costs) and have the same identifier. In these embodiments, it may be difficult to automatically distinguish between the input data from the two hand controllers 106A, 106B, so the interface software 111 may prompt the user to manually identify the two hand controllers (e.g., by first pressing a button on the left-hand controller 106A and then pressing a button on the right hand controller 106B.) This setting can then be stored by the interface software 111 as part of the device's software state representation.

The interface software 111 can use a similar process to distinguish input data from different hardware devices when multiple sets of hardware devices are connected to the host device 110 (e.g., for a multiplayer game). For example, the interface software 111 may display a prompt to move a first motion sensing device 104 (e.g., attached to a stationary bicycle), then display a prompt to move a second motion sensing device 104 (e.g., attached to an elliptical trainer), and then display similar prompts to identify the respective hand controllers 106 on the two fitness devices.

In one embodiment, signals from the hand controllers 106A, 106B and motion sensing device 104 are wirelessly sent to the host device 110 as input data using a custom Bluetooth GATT profile. The data sent from the hardware devices 102 are then processed by the host device API 112.

An example of the dataflow between the hardware devices 102 and the host device 110 is presented below:
1. Motion sensing device 104 acts as a server and the host device 110 connects as a client.
2. Host device 110 (the client) subscribes to data notifications of a specific service feature of the motion sensing device 104, such as an accelerometer.
3. A reading from the service feature (e.g., an accelerometer reading) is taken from motion sensing device 104.
4. The reading is sent to the host device 110 (the client subscriber). For example, the three-axis accelerometer reading may be sent as three bytes.
5. Interface software 111 decodes the reading and makes the readings available to applications 114 via the API 112. The readings are interpreted as input data by the application 114 and allow users to make selections or to control/manipulate elements (e.g., objects, characters, etc.) within the application 114 or to react to game cues or other interactive elements within the application 114.

The fitness applications 114 are applications that receive input data from the hardware devices 102 through the host device API 112 and carry out some sort of user interaction based on the input data. For example, the fitness applications 114 can be exercise games or any non-game application that can interact with a fitness device in a meaningful way (e.g., an application 114 interact with hardware devices 102 mounted on a treadmill to simulate a walk through a park).

The applications 114 contain one or more objects. In one embodiment, the object is a graphical representation such as a car, a ball or a character. The object also exhibits a controlled behavior, such as the graphical representation of the ball may exhibit a behavior of rolling. As the user performs the fitness activity the graphical representation or the object exhibits a controlled behavior based on the fitness activity performed by the user. For example, in one application the object can be a character representing the user. The character may exhibit a controlled behavior of running across the screen. The controlled behavior of running across the screen may be related to the speed at which the user runs. The faster the user runs the faster the character runs across the screen. Hence, the system allows the user to change and control the behavior of the object based on, in one instance, the intensity at which a user performs the fitness activity.

In another example the object is a set of numbers on a scoreboard. The number may represent a number of metrics, such as distance run by the user or the number of calories burned by the user. The numbers on the scoreboard exhibit a controlled behavior of changing at a rate related to the speed at which the user is running at. Hence the faster the user runs the rate at which the numbers on the scoreboard change increases.

In one embodiment, the input data received by the application 114 from the hardware devices 102 affects the behavior applied to the object in an application. Returning to the example of a character representing the user running across the screen; the user may press the button on the hand controller 106 causing the jumping behavior to be applied to the user's character in the application 114. This results in the user's character jumping over an obstacle as the user is playing the game. Hence, the user may also use the hardware devices 102 to apply a behavior to an object of the application 114.

Applications 114 (including games) may be configured to encourage the user to exercise. As the user exercises, data received from the hardware devices 102 is recorded on the host device 110 to reflect progress in the user's experience. Applications 114 can also connect to a fitness server 118 through the network 116, where user data and statistics can be stored and accessed. One example of how the hardware devices 102 can interact with the software on the host device 110 could be a user attaching the motion sensing device 104 to the pedal of a stationary bicycle. An accelerometer in the motion sensing device 104 measures the motion of the pedals, allowing for realistic control of a ‘virtual biking’ application 114. Another example of how hardware devices 102 interact with software could be a user attaching the motion sensing device 104 to their hip, with the motion sensing device 104 detecting that motion for a dance or exercise game.

The host device API 112 may be configured to run on any suitable operating system, such as iOS, Windows/Mac/Linux or Android. Different versions of the host device API 112 may be created for different operating systems. Although shown as a separate entity within the host device 110, the host device API 112 is typically a separate code library that is compiled into a fitness application 114 at build time. This allows a third-party developer can write a fitness application 114 that uses functions in the host device API 112 to exchange data with the hardware devices 102 and the fitness server 118.

In one embodiment, the fitness server 118 is comprised of a cloud services module 120, a fitness database 122 and an application database 124. The fitness server 118 and its components 120, 122, 124 may be embodied as a single device or as a cluster of networked devices (e.g., a cluster of web servers). In one embodiment, the cloud services module 120 provides an interface over the network 116, by which the host device 110 can access fitness data from the fitness database 122 and application data from the application database on the fitness server 118. The cloud services module 120 receives historical data, such as physical activity and game performance data, from the host device API 112 and stores the data in the appropriate database 122, 124. For example, on installation of the fitness application 114 on a host device 110 running an Android operating system, the fitness application 114 may have to retrieve Android specific configuration and system commands from the application database 124 on the fitness server 118. In another example, the interface software 111 stores fitness data with respect to a specific user in the fitness database 122 on the fitness server 118. The user can now access the fitness data 122 on multiple host devices 110.

In one embodiment, the cloud services module 120 also allows users to access their information on any device and at any time. The module also processes user requests to compare and share scores on third-party servers 126, such as social networking systems, e.g., FACEBOOK™ and TWITTER™. The databases 122, 124 may be stored across a cluster of several servers for scalability and fault-tolerance.

The cloud services module 120 can also host multiplayer games. In one embodiment, games are hosted in asynchronous context. After a user set ups a challenge, the cloud services module 120 can give a user a window in time to finish his “tum” or just wait until he completes a given goal. This information is stored in the application database 124. The cloud services module 120 executes the logic of the games, challenges, or goals that are created between players and maintains their states and progression.

In an alternative embodiment, the cloud services module 120 also hosts multiplayer games in real-time, with real-time game data moving back and forth between players on different host devices 110.

In another embodiment, a third party server 126 may communicate with the host device 110 or the fitness server 118 over the network 116. For example, the cloud services module 120 retrieves a user's social graph from a social networking system on a third-party server 126 and reproduces the social graph on the fitness server 118. Thus, subsequent functions associated with social game mechanics (e.g., the creation of challenges and cooperative goals) can be executed on the fitness server 118.

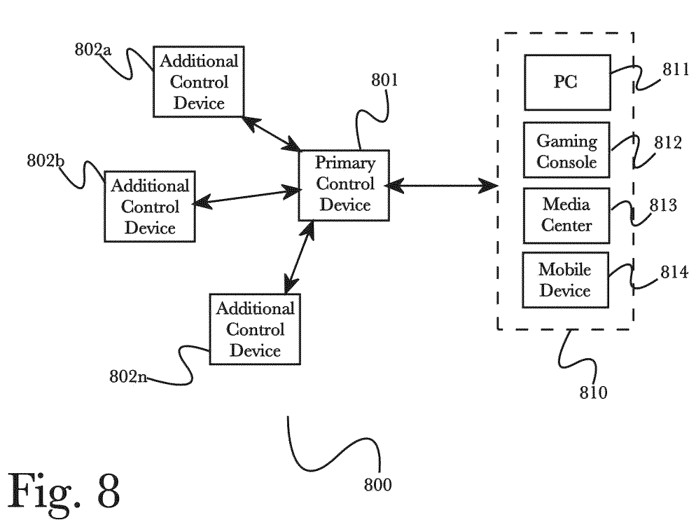

FIG. 8 is a block diagram of an exemplary system architecture 800, illustrating the use of nested-communication control devices 801, 802a-n with a variety of electronic systems 810, according to a preferred embodiment of the invention. According to the embodiment, a control device 801 may be connected to a plurality of electronic systems 810 such as including (but not limited to) a personal computer 811, video gaming console 812, media center (for example, a home theater system) 813, or mobile device 814 (for example, a smartphone or tablet computing device). Connection may occur via a variety of means, but according to the embodiment and as envisioned by the inventors, an ideal method of connection is via wireless connectivity means over the air. Exemplary communication protocols or technologies for such connectivity may include (but are not limited to) cellular radio communication, BLUETOOTH™, ANT™, WiFi, near-field communication (NFC), or other connectivity means.

According to the embodiment, a plurality of additional control devices 802a-n may be paired with a primary control device 801 via a variety of connectivity means. When connected in this manner, input from a user interacting via an additional control device 802a-n may be transmitted to primary control device 801, which may then convey the interaction to a plurality of connected electronic systems 810. Feedback, if provided by an electronic system 810, may then be received by primary control device 801 and conveyed to additional control devices 802a-n as needed (for example, if a user interacts via a particular additional control device 802a, and an electronic system 810 provides feedback intended specifically for that user or that device). It should be appreciated that not all control devices need to utilize similar connectivity means. For example, if a primary control device 801 is operating both a BLUETOOTH™ and a WiFi radio for wireless communication, an additional control device 802a may connect via BLUETOOTH™ while a second additional control device 802b may connect using WiFi. In this manner, a primary control device 801 may be considered a means to unify various connectivity means to facilitate communication between a plurality of electronic systems 810 and a plurality of additional control devices 802a-n of varying design and operation.

According to various aspects of the invention, control device 801 or any additional control devices 802a-n may be a fitness device of varying construction and capability; for example, control device 801 may be a treadmill, stationary bike, or elliptical trainer that is used as a primary control device to provide input data to a plurality of electronic systems 810, such that the fitness device (in this example, an exercise machine of varying construction) is used as a controller for user input.

Additional control devices 802a-n may, in this example, comprise a variety of fitness devices that are commonly used to connect to an exercise machine, such as (for example, including but not limited to) heart rate sensors, galvanic skin response (GSR) sensors, smartwatches, or other devices that may be used to produce relevant input data (such as from onboard hardware sensors within a device, for example an accelerometer or heart rate sensor).

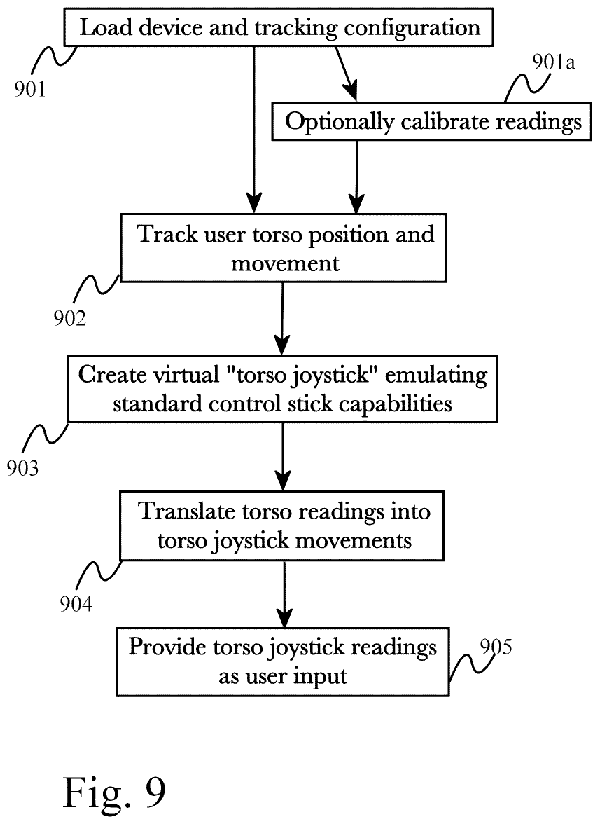

FIG. 9 is a block diagram of an exemplary system architecture of an exercise machine 900 being connected over local connections to a smartphone or computing device 930, an output device other than a phone 910, and a server 940 over a network 920, according to a preferred aspect. An exercise machine 100 may connect over a network 920, which may be the Internet, a local area connection, or some other network used for digital communication between devices, to a server 940. Such connection may allow for two-way communication between a server 940 and an exercise machine 800. An exercise machine 100 may also be connected over a network 920 to a smartphone or computing device 930, or may be connected directly to a smartphone or computing device 930 either physically or wirelessly such as with Bluetooth connections. An exercise machine 100 also may be connected to an output device 910 which may display graphical output from software executed on an exercise machine 100, including Mixed or virtual reality software, and this device may be different from a smartphone or computing device 930 or in some implementations may in fact be a smartphone or computing device 930. A remote server 940 may contain a data store 941, and a user verification component 942, which may contain typical components in the art used for verifying a user's identity from a phone connection or device connection, such as device ID from a smartphone or computing device or logging in with a user's social media account.

FIG. 2A is a block diagram illustrating the components of the motion sensing device 104, according to one example embodiment. In this embodiment, the motion sensing device 104 includes sensors 202, a communication module 208, and firmware 210. However additional or different sensors 202 may be included in the motion sensing device 104. In one embodiment the user places the motion sensing device 104 on their body, such as on their arm, wrist, leg or ankle. In another embodiment, the user places the motion sensing device on the fitness system, such as on the handlebars of an elliptical machine.

The sensors 202 generate motion data based on an activity performed by the user. The activity performed by the user may include, running, cycling, using the elliptical machine, or any other form of exercise. In one embodiment the sensors 202 may include an accelerometer 204 and a gyroscope 206. In one embodiment, the accelerometer 204 captures acceleration data 204 based on the acceleration of the motion sensing device 104 along multiple axes. In one embodiment, the gyroscope 206 captures a rate of rotation of the motion sensing device 104 along multiple axes.

In one embodiment, the firmware 210 in the motion sensing device 104 is a combination of a persistent memory, a program stored in the memory and a set of hardware required to execute the program. The firmware 210 retrieves, formats and temporarily stores the data output by the accelerometer 204 and the gyroscope 206. In other embodiments the firmware 210 may perform other activities, such as controlling the rate at which data is retrieved from the accelerometer 204 and the gyroscope 206 or averaging samples of data retrieved from the accelerometer 204 and the gyroscope 206.

In another embodiment, the firmware 210 contains logic that manages the operation of the motion sensing device 104. For example, the firmware 210 may contain logic to support a “shake to connect” function that monitors the sensors 202 and establishes the communication link 108A to the host device 110 upon detecting that a user has shaken the motion sensing device 104.

In an alternative embodiment described with reference to FIG. 1, the two hand controllers 106A, 106B are coupled to the motion sensing device 104 and the motion sensing device 104 is configured to relay input data from the hand controllers 106A, 106B to the host device 110. In this alternative embodiment, the firmware 210 is configured to detect and connect to the hand controllers 106A, 106B independently of the host device 110, and the communication module 208 maintains the connections with the hand controllers 106A, 106B after they are established. The connections with the hand controllers 106A, 106B may be under the same communication protocol as the communication link 108A (e.g., Bluetooth Low Energy) or a different communication protocol (e.g., an RF connection). Meanwhile, the firmware 210 manages the three connections (the connections with the two hand controllers 106A, 106B and the communication link 108A with the host device 110) and relays input data from the hand controllers 106A, 106B to the host device 110. In some embodiments, the firmware 210 may be configured to combine the input data from the sensors 202 and the hand controllers 106A, 106B into a single integrated input stream. Thus, the host device API 112 receives a single input stream that includes inputs from all three hardware devices.

In one embodiment, the communication module 208 transmits the formatted acceleration and rate of rotation data from the firmware 210 to the host device 110. The communication module 208 interacts with the communication link 108A to transfer data between the motion sensing device 104 and the host device 110. In one embodiment, the communication module 208 connects via the Bluetooth Low Energy (BLE) protocol.

FIG. 2B illustrates a motion sensing device 104, according to another example embodiment. In this embodiment, the motion sensing device 104 is comprised of an electronic circuit 213, and an outer shell 212. The outer shell 212 houses the electronic circuit 213. The outer shell also contains an attachment mechanism to attach the motion sensing device 104 to the user or the fitness system. For example, the user can attach the motion sensing device 104 to their wrist, ankle or to the handle bars on an elliptical machine via the attachment mechanism. The electronic circuit is comprised of the accelerometer 204, the gyroscope 206, the communication module 208 and firmware 210. In the illustrated embodiment, the motion sensing device 104 connects wirelessly to the host device 110 and is powered by a battery.

FIG. 2C illustrates a hand controller 106, according to one example embodiment. In this embodiment the hand controller 106 is comprised of an attaching mechanism 216, a hand controller shell 214 and buttons 218. Apart from buttons 218 the hand controller may also include a directional pad or joystick either in addition to or in place of the buttons 218. As described above with reference to FIG. 1, the hand controller 106 communicates with the host device 110. In the illustrated embodiment, the hand controller 106 communicates wirelessly and is battery powered.

In one embodiment, the attaching mechanism 216 is used to attach the hand controller to the fitness system or the user. For example, the attaching mechanism 216 is a strap used to strap the hand controller 106 onto the handlebar of an elliptical machine. In other embodiments, the attaching mechanism is not limited to a strap. In one embodiment, the hand controller shell 214 houses the electronic components of the hand controller 106 and other relevant components such as a battery. The form factor of the hand controller shell 214 facilitates the attaching of the hand controller 106 to the user or the fitness system. In one embodiment, the buttons 218 on the hand controller 106 receive user input.

Internally, the hand controller 106 includes a radio and a microcontroller. In one embodiment, the radio is an RF radio that connects to the motion sensing device 104 and sends an input signal to the motion sensing device 104 to be relayed to the host device 110. In an alternative embodiment, the hand controller includes a Bluetooth Low Energy (BLE) radio and a system on a chip (SoC).In some embodiments, the radio, microcontroller, and any other electronic components of the hand controller 106 may be placed on a single printed circuit board.

FIG. 2D illustrates an example of hand controllers 106A, 106B mounted to handlebars of a fitness device, according to one embodiment. The straps 216 of the hand controllers 106A, 106B are wrapped around the handlebars. In one embodiment, the bottom side of the hand controller shell 214 may include an adhesive substance to increase the friction between the hand controllers 106A, 106B and the handlebars.

To reduce manufacturing costs, some or all of the components of the two hand controllers 106A, 106B may be identical. In one embodiment, the two hand controllers 106A, 106B are completely identical (e.g., same mechanical enclosure, internal electronics, and firmware). In this embodiment, it may be difficult for the firmware 210 or the host device API 112 to distinguish between the two hand controllers, and the interface software 111 may prompt the user to identify each hand controller as described above with reference to FIG. 1. In another embodiment, the two hand controllers have the same internal electronics (e.g., the same microcontroller, radio, etc.), but may be placed in different enclosures to identify one controller as the left hand controller 106A and the other as the right hand controller 106B. For example, the buttons 218 on the two hand controllers may have a different layout or different colors, or the housings may have symmetric ergonomic features so that they are better suited for a user's hands. The two hand controllers 106 may also be flashed with different firmware so that the motion sensing device 104 or host device API 112 can distinguish between the input data from the two controllers 106. For example, the firmware on the left hand controller 106A may be configured to send an identifier (e.g., a binary 0) in a header of its input stream, while the firmware on the second hand controller 106B may be configured to send a different identifier (e.g., a binary 1) in a header of its input stream.

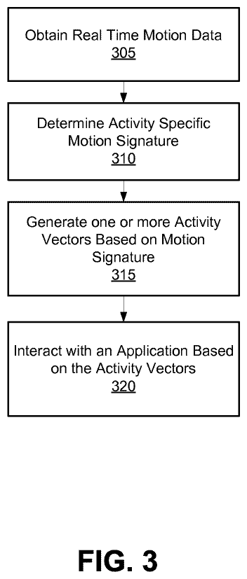

FIG. 3 is a flowchart illustrating an example process for interacting with a fitness application 114 based on the user's interaction with the fitness system, according to one embodiment. The user begins by attaching the hardware devices 102 to a fitness device or to the user's body; the hand controllers 106A, 106B can either be held in the user's hands or attached to the fitness device, and the motion sensing device 104 may be attached to the user's body or to the fitness device, depending on which fitness application 114 the user selects. One of the fitness applications 114 may be an instruction application that displays instructions for mounting the hardware devices 102 on a selected fitness device.

In one embodiment, the motion sensing device 104 obtains 305 real time motion data comprised of acceleration data from the accelerometer 204 and rotation data from the gyroscope 206, as the user interacts with the fitness system. The fitness system is not limited to a stationary exercise machine such as a treadmill, but may also include dynamic means of exercise, such as running on the road. The data obtained by the motion sensing device is 104 is transmitted to the host device 110 over established communication links 108.

Based on the data obtained 305, the interface software 111 on the host device determines 310 an activity specific motion signature. The activity specific motion signature identifies the current activity performed by the user. In one embodiment, the motion signature acts as a basis against which future movements performed by the user are compared against by the fitness applications 114.

In one embodiment, the interface software 111 generates one or more activity vectors 315 based on the motion signature. The activity vectors represent metrics associated with the activity level of the user. For example, the activity vectors may represent the number of calories burned by a user, the distance covered over a specific time interval or the intensity level of the user. The activity vectors are a standard unit of measurement across different types of fitness devices.

In one embodiment, the user interacts 320 with the fitness application 114 based on the activity vector generated 315 by the interface software 111. By altering the intensity with which a user performs a fitness activity, the user changes the activity vector over a period of time. The change in the activity vector alters the state of an object in the fitness application 114, or the behavior of the object in the fitness application 114 thereby allowing the user to interact with the fitness application 114 on the host device 110. For example, if the user is playing a running video game, where the user's character is running on the screen through an obstacle course, the intensity at which the user runs on the treadmill or cycles on a bicycle influences the speed at which the user's character (the object in an application 114) runs across the screen (behavior exhibited by the object) in the video game. Thus by running faster, the user changes the activity vector in real time, corresponding to an increase in speed of the user's character. The user may also interact with the application based on input from the hand controllers 106. For example, the user could press a button on the hand controller 106 as the user's character approaches an obstacle in the video game, causing the user's character to jump over or avoid the obstacle.

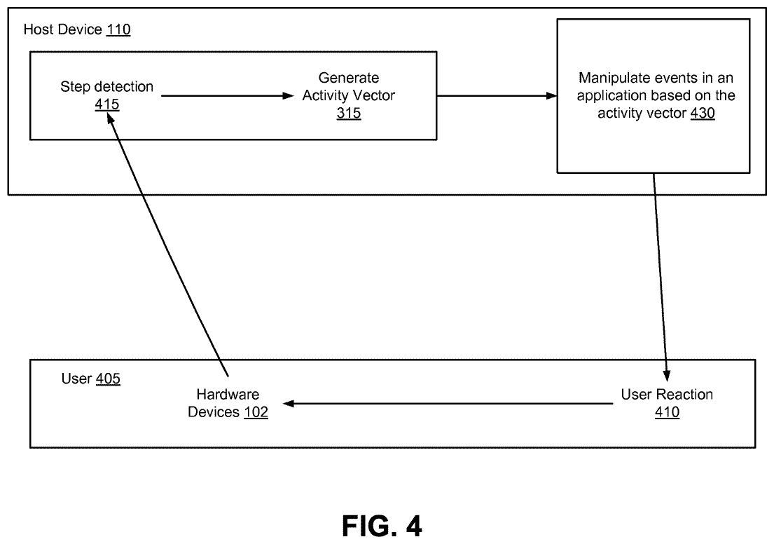

FIG. 4 illustrates an example process of a user 405 manipulating events in a fitness application 114 based on the user's 405 interaction with the fitness system in real time, according to one embodiment. In this embodiment, the events in a fitness application 114 may influence a user reaction 410. The user reaction 410 is any change in user activity or intensity of the fitness activity performed by the user 405. For example, the user 405 may notice that the user's character in a video game is not running fast enough to avoid a disaster. This may generate the user reaction 410, where the user 405 begins to run faster while running on a treadmill. In other, embodiments the user 405 may make no change to the intensity at which the user 405 performs a fitness activity.

The hardware devices 102 capture motion data and input data to the hand controllers as the user 405 performs the fitness activity. In one embodiment, the user reaction 410 generates an increase in the magnitude of the motion data captured by the hardware devices 102, as compared to previous motion data captured by the hardware devices 102. The hardware devices 102 transmit the captured data to the host device 110 over communication links 108.

The interface software 111 on the host device 110 processes the motion data captured by the hardware devices 102. In one embodiment, the interface software 111, executes a step detection 415 function, to determine the steps taken by a user 405 over a time period. The step detection is performed using the activity specific motion signature 310 determined by the interface software 111, as a basis. In one embodiment, a step represents a unit of movement of the fitness activity performed by the user 405. For example, a single step while walking and running, or a single rotation of a bicycle pedal while cycling would be detected 415 as a single step by the interface software 111.

In one embodiment, the interface software 111 generates an activity vector 345 based on the number of steps over a time window, or a time span over which a single step takes place. The motion signature of the activity performed by the user 405, determines how the number of steps over a specific time window, or the time between two steps influences the value of the activity vector. For example, one revolution on a bicycle may be considered as 2 steps while running. Thus, the interface software 111, identifies what activity a step is related to based on the motion signature determined by the interface software 111. Then the interface software 111 modifies or generates the activity vector based on the number of steps in a given time window, or the time between steps and the motion signature associated with the step. This allows the user 405 to switch between fitness machines, such as move from running on a treadmill to cycling on a fitness bicycle, but still generate consistent activity vectors representing the intensity at which the user 405 performs different fitness activities across different fitness machines.

In one embodiment, the number of steps within a given time window represents the motion signature associated with a fitness activity. For example, running may correspond to fewer steps being detected in the given time window as compared to bicycling. In another embodiment, the activity vector is an intensity level that is scaled inversely to the time between steps for a specific motion signature. For example, if the time between steps is 0.5 seconds the intensity level is given a value of 5 (on a scale of 0 to 10). However, when the time between steps reduces to 0.4, the intensity level is given a value of 7 (on a scale of 0 to 10). In a different embodiment, the activity vector is an intensity level inversely to the number of steps in a given time window.

Based on the activity vector the user 405 manipulates events in the fitness application 114 in real time. In one embodiment, elements or objects in the fitness application 114 are given attributes based on the activity vector generated 315 by the interface software 111. For example, the fitness application 114 may be a game, where the rate at which points are earned by the user 405 increases as the magnitude of the activity vector increases. Hence, the user 405 may run faster on a treadmill to increase the number of steps per given time window, thereby increasing the magnitude of the generated activity vector, manipulating 430 the rate at which points are increased in the game.

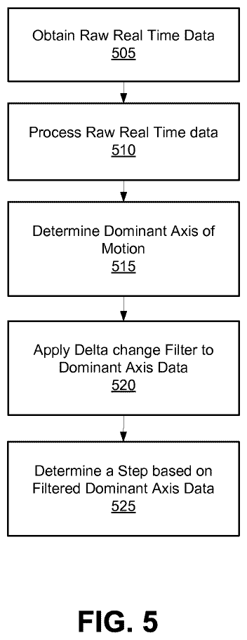

FIG. 5 is a flow chart illustrating an example process for determining a step taken by the user, based on motion data, according to one embodiment. The hardware devices 102 obtain 505 raw real time motion data as the user performs the fitness activity. The hardware devices 102 transmit the raw motion data to the host device 110 and interface software 111 over communication links 108. In one embodiment, the interface software 111 processes 510 the raw motion data. There are a number of techniques that may be applied to process 510 the raw motion data. For example, the interface software may use a high pass filter 510 to process the raw motion data on each axis of motion or rotation. The high pass filter attenuates or reduces the magnitude of data with frequencies below a cutoff frequency.

In one embodiment, the interface software 111 determines 515 a dominant axis of the processed motion data. The dominant axis represents the axis along which there is most motion captured by the hardware devices 102, as the user performs the fitness activity. Determining 515 the dominant axis of motion, helps the interface software 111 identify the primary direction or axis of motion of the user as the user performs a fitness activity. In one embodiment, the primary direction of motion of the user influences at least in part the motion signature generated by the interface software 111.

In one embodiment, the dominant axis is determined 515 by summing the absolute value of each axis of processed motion data over a rolling time window. In one embodiment, the axis with the largest absolute value over the rolling time window is picked as the dominant axis of motion. If a good set of data is not obtained for the current dominant axis, the dominant axis is changed to the axis with the next largest absolute value. In one embodiment, a good set of data is determined by comparing the ratio of the absolute value of the current dominant axis to the sum of the absolute values of the other axes, and a threshold value.

In one embodiment, a delta change filter is applied 520 to the dominant axis of the processed 510 motion data. The delta change filter increases or decreases the difference between a current value and in a time series of motion data and a previous value in the time series. For example, if the current value of the processed motion data is similar to the previous value in the time series, the delta change filter changes the current value in the time series to equal that of the previous value. If the current value is significantly different from the previous value the delta change filter changes the current value by an interpolated amount so that the difference between the current value and the previous value increases (i.e., the absolute difference).

In one embodiment, the interface software 111 determines 525 the step taken by the user based on the time series of the processed data along the dominant axis of motion. For example, a step is detected 525 when there is a sign change in the acceleration data of the determined 520 dominant axis. In another embodiment, an acceleration threshold must be crossed just before the sign change in the acceleration data, for the step to be considered a potential step. For example, the time series acceleration value must be greater than 0.5 G, before a sign change is identified in a time window containing 20 accelerometer samples collected over 333 milliseconds (ms).

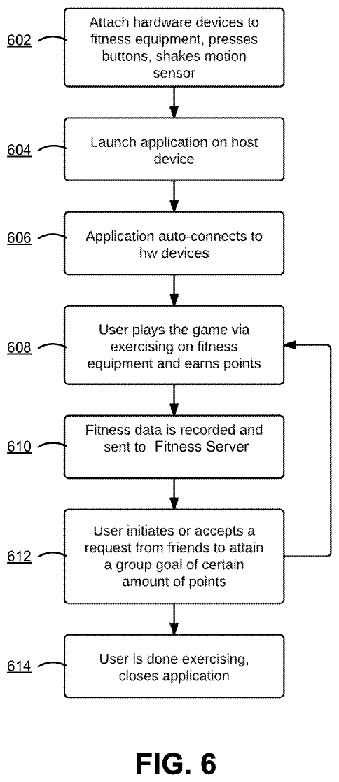

FIG. 6 is a flow chart illustrating an example process for interacting with the adaptable fitness system, according to one embodiment. The user begins by attaching 602 the hardware devices 102 to a fitness device or to the user's body; the hand controllers 106A, 106B can either be held in the user's hands or attached to the fitness device, and the motion sensing device 104 may be attached to the user's body or to the fitness device, depending on which fitness application 114 the user selects. One of the fitness applications 114 may be an instruction application that displays instructions for mounting the hardware devices 102 on a selected fitness device.

Next, the user selects and launches 604 a fitness application 114, and the host device API 112 initiates a search to auto-connect 606 with the hardware devices 102, which runs in the background. The application 114 provides a user interface indicator showing the state in which the hardware devices 102 are connecting. The user then begins to interact 608 with the application 114. As the user works out, the motion sensing device 104 sends input data to the application 114 tracking player movement. The hand controllers 106A, 106B send input data to the application tracking player game inputs. The application 114 shows the user game cues and feedback. The operating system of the host device 110 runs the application.

The host device 110 records 610 fitness data for the user to review. The fitness data is reflected in quantifiers such as steps taken and calories burned. The user is encouraged to exercise by seeing his game progression and fitness data. The application 114 may optionally use the host device API 112 to send the fitness data to the fitness server 118 to be saved. If the host device 110 does not have network connectivity at the time the fitness data is recorded 610, then the data may be automatically sent when network connectivity becomes available. If the user chooses to use the fitness application 114 anonymously (i.e., without logging in to a user account), then the user's session is tracked anonymously.

The user is also able to share his game progression and fitness data online with friends on social networking systems, which provides further encouragement. In addition, the user may use social features of the fitness server 118, such as initiating/accepting requests 612 to attain a group goal (e.g., a specified number of movement units) or initiating/accepting challenges to attain an individual goal. If a request or challenge is activated, then the user begins interacting 508 with the application 114 again. When the user is done, the user ends the session by closing 614 the application 114.

In one embodiment, the user can create a personal goal to achieve an activity vector value in a given time period. For example, a goal can be to run 50 miles in one week, while maintaining an intensity level of 7 (based on an intensity level scale ranging from 0 to 10). These personal goals can then be shared with friends within a social network.

Users can create also cooperative goals for a group in a social network to achieve an activity vector value. For example, a group of five people may set a goal to achieve 250 miles for the week, while all maintaining the intensity level of 7.

Activity vectors can also be used as a basis for activities that benefit from normalization of data for comparison purposes. For example, activity vectors can be used in competitions between groups or friends in a network. Challenges can be issued and broadcast to a user's friends on social network.

FIG. 9 is a flow diagram illustrating an exemplary method for natural body interaction for mixed or virtual reality applications, according to a preferred embodiment of the invention. In an initial step 901, a control device 801 (as described previously, referring to FIG. 8) may load a variety of device and tracking configuration data, such as preconfigured parameters to establish a “default mode” or baseline tracking behavior, or using historical data from previous sessions if available. Device tracking may optionally be calibrated 901a if needed, for example if new devices are detected or an arrangement has changed since previous operation, or if a user manually requests calibration (or any other criteria, such as a configured calibration time interval). Devices may then begin tracking a user's torso position and movement 902, providing these readings to the composition server as input data. In a next step 903, control device 801 may create a virtual “torso joystick” within a software application, to emulate a standard control stick input device in software without needing a hardware device present. In a next step 904, control device 801 may translate received torso readings from previous steps into movements of the software-based torso joystick, and in a final step 905 may provide these torso joystick movements as user input for further use. In this manner, movement of a user's body may be used to emulate the movements or other behaviors of a control stick, enabling complex and reliable interaction with software applications through natural body movements. By translating these movements into joystick input via control device 801, this functionality may be added to existing software programs and games that have support for control stick interfaces, without the need for additional configuration.

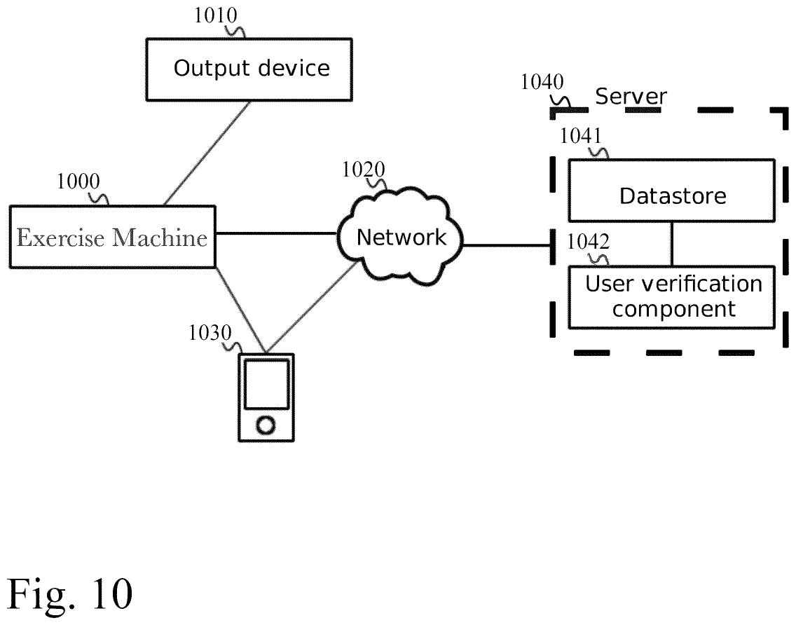

FIG. 10 is a block diagram of an exemplary system architecture of an exercise machine 1000 being connected over local connections to a smartphone or computing device 1030, an output device other than a phone 1010, and a server 1040 over a network 1020, according to a preferred aspect. An exercise machine 1000 may connect over a network 1020, which may be the Internet, a local area connection, or some other network used for digital communication between devices, to a server 1040. Such connection may allow for two-way communication between a server 1040 and an exercise machine 1000. An exercise machine 1000 may also be connected over a network 1020 to a smartphone or computing device 1030, or may be connected directly to a smartphone or computing device 930 either physically or wirelessly such as with Bluetooth connections. An exercise machine 1000 also may be connected to an output device 1010 which may display graphical output from software executed on an exercise machine 1000, including Mixed or virtual reality software, and this device may be different from a smartphone or computing device 930 or in some implementations may in fact be a smartphone or computing device 1030. A remote server 1040 may contain a data store 1041, and a user verification component 1042, which may contain typical components in the art used for verifying a user's identity from a phone connection or device connection, such as device ID from a smartphone or computing device or logging in with a user's social media account.

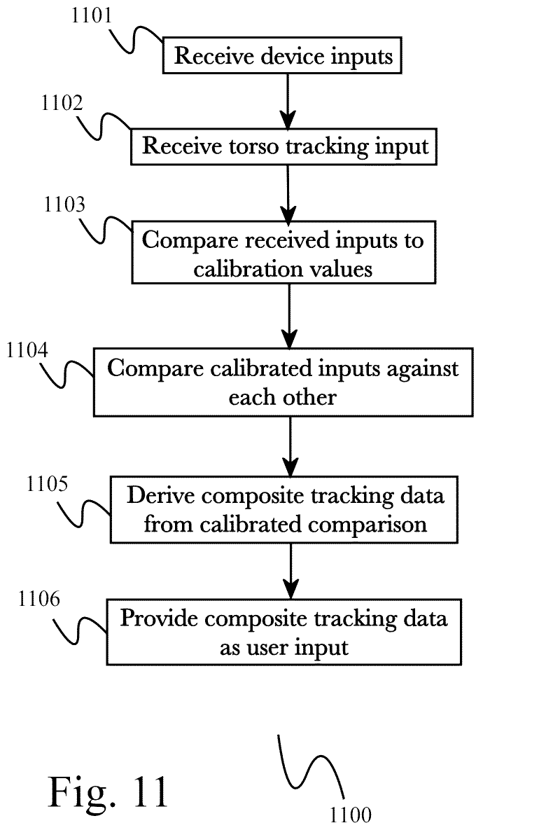

FIG. 11 is a flow diagram illustrating an exemplary method 1100 for processing natural body interaction and additional inputs and producing a composite output, according to a preferred embodiment of the invention. In an initial step 1101, a control device 801 may receive a plurality of device inputs, such as motion data from a headset, button inputs, or fitness data from any of a variety of fitness tracking devices such as fitness-enabled smartwatches or biometric sensors, or other device input. In a next step 1102, control device 801 may receive a variety of torso tracking input, such as movement data from a headset or torso position or movement tracking via a plurality of sensors. In a next step 1103, received data may be compared to calibration values to perform data “clean up”, for example by discarding erroneous readings or by adjusting readings based on known calibration (such as applying an offset to normalize readings), and in a next step 1104 the resulting calibrated readings may be further compared against each other and further refined as necessary (for example, applying an offset or bias to a portion of readings to normalize them relative to other readings, such as having an “axis multiplier” to correct for distorted movement along a particular axis relative to other axes). In a next step 1105, these calibrated readings may then be used to derive composite tracking data, such as by utilizing tracking of hands and head to identify complex movements of a user's hands relative to their face, or by combining head and torso movement to identify more complex poses or movements of the user's body, such as leaning in one direction while looking in another, or attempting to hold a specific complex pose such as for yoga or contortion-based games. In a final step 1106, composite data may be provided as user input for further use in software applications, for example for use in a gaming application or for use by a connected computing device such as a personal computer or video game console. In this manner, multiple data type or sources may be used to derive more complex and detailed movements and other data, and this may be combined into a single composite input for use in software applications according to their particular configuration (such as for use in a video games designed to accept control stick input, but not designed or readily adaptable to utilize fitness tracker data).

### Exemplary Controller Configurations

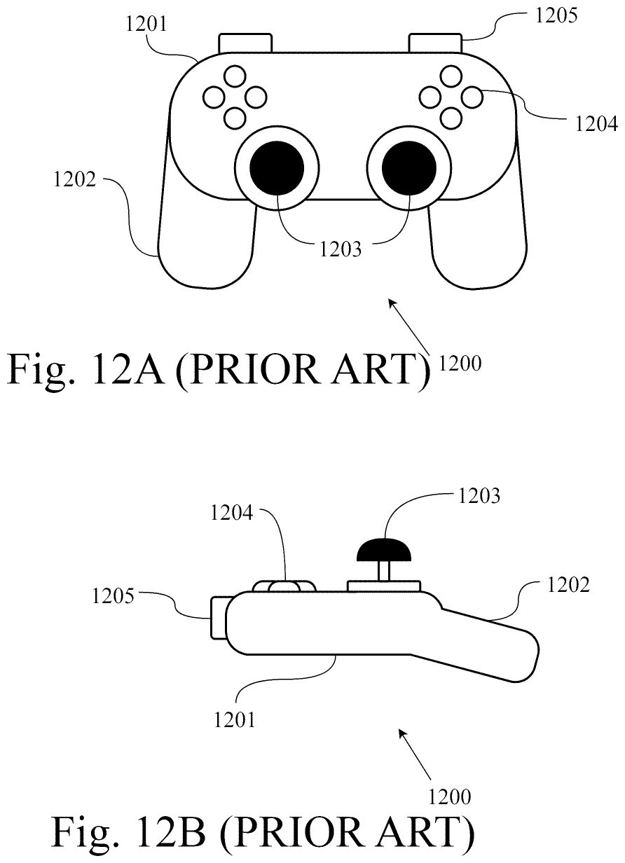

FIGS. 12A & 12B (PRIOR ART) show a representation of a standard dual joystick controller 1200. FIG. 12A (PRIOR ART) shows a top view and FIG. 12B (PRIOR ART) shows a side view of such a controller. A standard dual joystick controller, such as a DUALSHOCK™ controller or an XBOX ONE™ controller, has a single-piece body 1201 comprising handles 1202, two small joysticks 1203 designed to be controlled with the thumbs of each hand, a plurality of buttons 1204 which are typically located in front of each joystick, and a plurality of trigger buttons 1205 which are typically mounted on the front of the dual joystick controller to mimic triggers as one would find on a handgun or rifle. Dual joystick controllers are typically used while sitting, and require fine motor skill operation of the hands and fingers. Thus, whole body movements interfere with the use of such controllers because the fine motor skills required to operate the controllers are over-ridden or thrown off by gross body movements, particularly vigorous body movements like those that occur during exercise. Accordingly, dual joystick controllers are typically used while stationary, and most often while sitting.

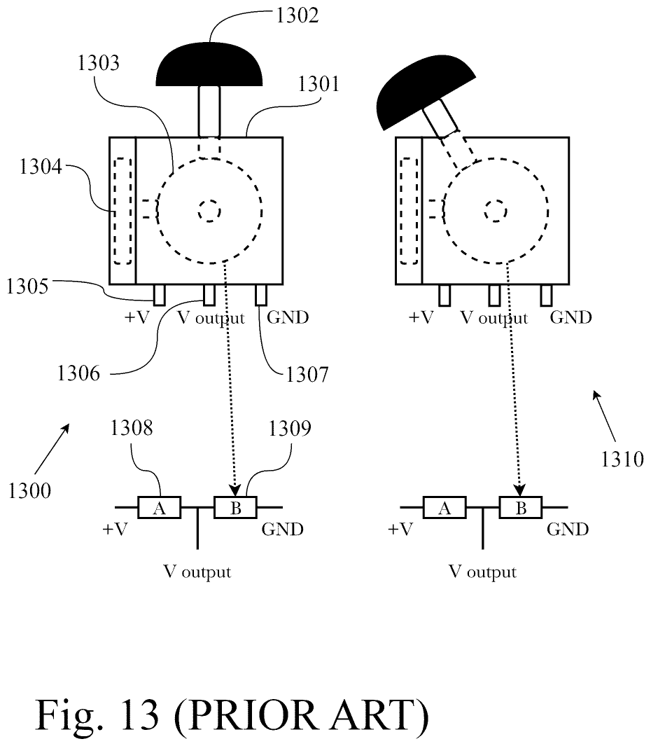

FIG. 13 (PRIOR ART) shows the design and electrical characteristics of a typical thumb joystick as found in a standard dual joystick controller. While a typical thumb joystick is shown in this example, this design is fairly standard across all modern joysticks. The thumb joystick controller comprises a housing 1301 made of rigid material, typically plastic, a joystick with a mushroom-shaped top 1302 typically made of a rubberized material for grip, a first potentiometer 1303, 1309 mounted parallel with the x-axis of the joystick, and a second potentiometer 1304 mounted parallel with the y-axis of the joystick (i.e., perpendicular to the first potentiometer), a reference voltage pin 1305, an output voltage pin 1306, and a ground pin 1307. In the typical usage, a +3.3V to +5V voltage (typical voltages used in computer systems) is applied to the reference voltage pin. The three pins 1305, 1306, and 1307, along with a fixed resistor 1308, and the potentiometer 1303, 1309, act as a voltage splitter. The fixed resistor 1308 and potentiometer are matched, such that when the joystick is at rest 1300, each resistor drops about half of the voltage applied at the reference voltage pin. For example, for a reference voltage of +5V a fixed resistor of 10KΩ with the potentiometer at rest also of 10KΩ, the voltage would be split equally between the fixed resistor 1308 and the potentiometer 1303, 1309 such that voltage at the output voltage pin 1306 would be 2.5V. When the joystick is moved on the x-axis 1310, the resistance of the potentiometer is either increased (pushing more voltage to the output voltage pin 1306) or decreased (passing more voltage through to the ground pin 1308, changing the relationship with the fixed resistor and changing the voltage available at the output voltage pin 1306. The same is true of the y-axis, and the different voltages from the x-axis and y-axis are used to determine the location of the joystick. The analog voltages (also known as analog signals) from the x-axis and y-axis are typically converted to digital form (also known as digital signals) using an analog-to-digital converter, typically with an 8-bit or 10-bit resolution, corresponding to a digital value on each axis between 0-255 or 0-1023. is then used for proportional control of a device. The trigger buttons on dual joystick controllers are typically proportional, as are the joysticks, whereas the other buttons on the face of the controller are usually on/off (i.e., binary or 1/0) buttons.

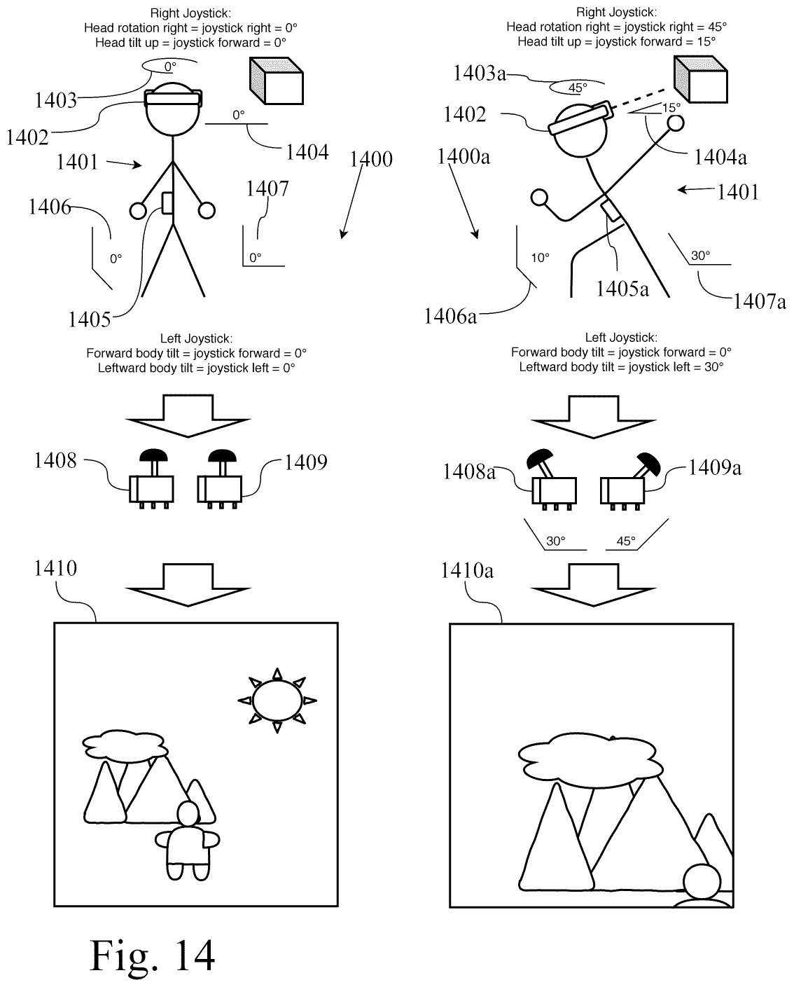

FIG. 14 shows an exemplary embodiment and the relationship between the motion sensors, standard dual joystick controllers, and the resulting image that would be seen when using the embodiment to control a computer game. The user 1401 wears a motion sensor on the head 802 and a motion sensor worn on the torso 1405. In this example, the user 1401 is playing a first-person virtual reality game. The motion sensor worn on the head 1402 is configured to track both a left/right rotation of the head 1403 (corresponding to the user looking left or right) and an up/down tilt of the head 1404 (corresponding to the user 1401 looking up or down). The motion sensor worn on the torso 1405 is configured to track a left/right torso lean 1406 (corresponding to the user 1401 leaning left or right) and a forward/backward torso tilt 1407 (corresponding to the user 1401 leaning forward or backward). The version on the left side 1400 shows the user 1401 standing straight up, corresponding to both the left joystick 1408 and the right joystick 1409 at rest in the upright position, with an initial in-game image showing a scene 1410 containing a person, mountains, clouds, and a sun. The version on the right side 1400a shows the user interacting with the game by leaning and tilting his body/torso and by rotating and tilting his head. The user 1401 is leaning to the left at a 30° lean 1407a, and leaning forward at a 10° tilt 1406a. These body movements correspond to a left joystick lean of 30° 1408a and a left joystick forward tilt of 10° (not shown), causing the user's in-game avatar to move left (objects in image appear to shift to the right) and slightly forward (objects in image appear to become larger). Simultaneously, the user's head is rotated 45° to the right 1403a and 15° upward 1404a. These head movements correspond to a right joystick lean of 45° 1409a and a right joystick backward tilt (representing looking upward) of 10° (not shown), causing the user's in-game view 1410a (through the eyes of the in-game avatar) to move right (objects in image appear to shift to the left) and slightly upward (objects in image appear to move slightly downward). From this example, it can be seen that the user's 1401 perception of the virtual reality scene will depend on two separate sets of movements, one for the location of the in-game avatar's body being controlled by a virtual joystick corresponding to the user's 1401 torso, and a separate one for the in-game avatar's view being controlled by a virtual joystick corresponding to the user's head. The combination of these two virtual joysticks determines the in-game perspective and is equivalent to the use of dual joysticks on a standard dual joystick controller. It should be further recognized that embodiments are not limited to use in virtual reality or mixed reality gaming, and that embodiments may be used to operate any device that is controllable by joysticks. For example, dual joystick controllers are often used to operate radio-controlled drones, aircraft, and ground-based vehicles and machines, and some embodiments may be used to control these devices. Some embodiments may be used to operate devices that have other types of proportional controls, such as wheels, dials, variable pressure sensors, etc. For example, many radio controlled cars are operated by a proportional control trigger for which one virtual joystick could be substituted, and by a proportional control wheel or dial (imitating steering) for which a second virtual joystick could be substituted.

To convert the virtual joystick outputs to a standard joystick control signal, one simply needs to know the analog voltages and/or digital values accepted by the device to be controlled and map the virtual joystick outputs to those analog voltages and/or digital values. This mapping can be pre-programmed into the system or can be done automatically via software (e.g., the system can convert or map the virtual joystick outputs proportionally to the expected device inputs). The connection with the device to be controlled can be either wired or wireless. With wired connections, many devices use standard universal serial bus (USB) inputs, in which case digital values from the virtual joystick outputs can be transferred to the device to be controlled using standard USB hardware and protocols. In the case of wireless control, a connection is typically made via a Bluetooth™ or Wi-Fi™ adapter, and digital values are transferred wirelessly.

In some embodiments, the motion sensor worn on the head 1402 may comprise a viewing screen in the manner of a virtual reality headset. However, it is important to note that virtual reality headsets do not operate in the manner of the invention herein described. A virtual reality headset will contain motion sensors, but can only represent one of a plurality of joystick inputs. Further, the motion sensor worn on the head 1402 is not required to have a viewing screen, thus making it lighter and more compatible with the vigorous movements of exercise. In some embodiments, the motion sensors will not be worn on the head or body, and may be an externally-mounted devices such as a cameras that tracks head and body movements.

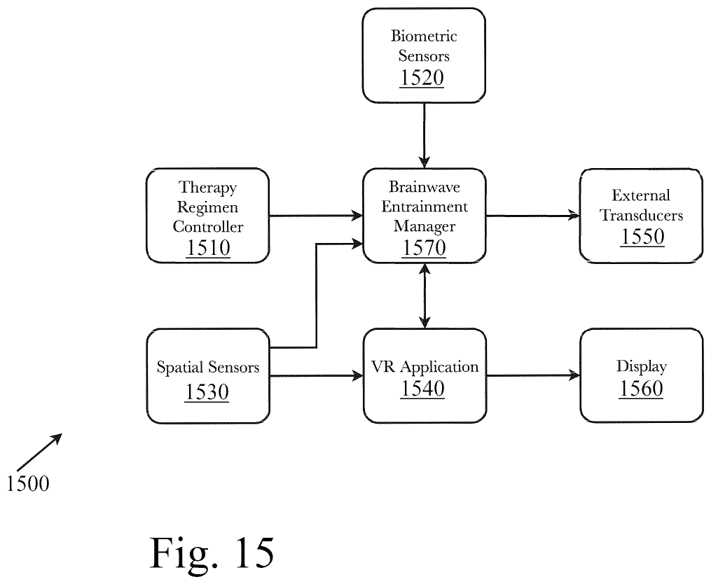

FIG. 15 is a diagram showing an exemplary overall system architecture 1500 for a brainwave entrainment system using virtual objects and environments as visual stimulation transducers. In this embodiment, the system comprises a brainwave entrainment manager 1570, a virtual reality (VR) application 1540, a therapy regimen controller 1510, one or more spatial sensors 1530, one or more biometric sensors 1520, and one or more external transducers, and a display 1560.

The brainwave entrainment manager 1570 is the core of the system, and manages inputs from, and outputs to, other components of the system. It is responsible for selection of entrainment routines, evaluation of the user's attention, and activation of both virtual and physical stimulation transducers.

The therapy regimen controller 1510 is an administrative interface that allows an administrator (e.g., a physician, therapist, masseuse, or other service provider) to select therapy regimens for application to the user (who may be a patient, client, etc., of the administrator). The therapy regimen controller 1510 may be used, for example, to select a regimen for brainwave entrainment that emphasizes alpha wave stimulation to induce relaxation in an overstimulated user.

The biometric sensors 1520 are sensors that measure a physical or physiological characteristic of the user, such as heart rate, temperature, sweat production, brain activity (using an electroencephalograph, or EEG), etc. Biometric sensors 1520 are used to provide feedback to the brainwave entrainment manager 1570 as to the physical or physiological state of the user, which may be used to infer the user's mental state. For example, a biometric sensor 1520 that measures the user's heart rate may be used to infer the user's level of relaxation (or lack thereof), thus providing feedback as to the effectiveness of alpha brainwave entrainment intended to induce relaxation.

Spatial sensors 1530 are sensors that measure a user's physical location in space or a location at which the user is focusing his or her attention. For two dimensional screens, eye movement may be tracked and the location of the user's gaze may be calculated. In the case of virtual reality (VR), the user's body may be tracked, or if the user is wearing a VR headset, the orientation of the headset can be used to detect the user's head movements. Spatial sensors 1530 are used to detect the user's engagement with virtual objects and virtual environments, such that brainwave entrainment using those objects and environments can be adjusted, accordingly.

The VR application 1540 is used for gamification of brainwave entrainment. While a VR application 1540 is shown here, in principle any computer game, puzzle, display, or animation can be used, whether interactive or not, and whether three-dimensional or two-dimensional. The VR application 1540 can be a specially-designed program intended for use with the system, or can be an off-the-shelf game or application adapted for use with the system. In either case, the VR application 1540 will either have an interface with the brainwave entrainment manager 1570, or will have a brainwave entrainment manager 1570 integrated into it, whereby the brainwave entrainment manager 1570 is used to control brainwave entrainment using the virtual objects in the VR application 1540.

The external transducers 1550 are physical stimulation transducers that may be used to complement brainwave entrainment using virtual objects. A non-limiting list of external transducers 1550 includes lights or LEDs, speakers or other audio-producing devices, vibratory or other pressure-producing devices, and electrical stimulators. As an example, while brainwave entrainment is being applied visually using virtual objects on a screen, the brainwave entrainment may be supplemented or complemented by audible brainwave entrainment using speakers.

The display 1560 may be any type of display producing an output visible to a user of the system. A non-limiting list of displays 1560 includes computer and tablet screens, VR headsets, and projectors. The display 1560 is the means by which visual brainwave entrainment may be applied using virtual objects.

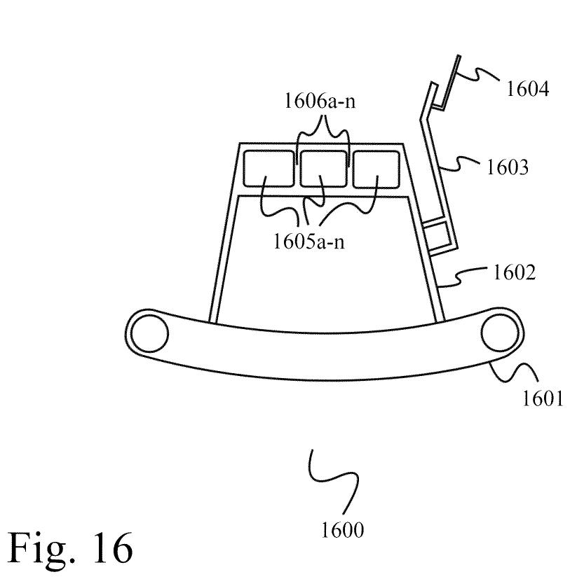

FIG. 16 is a side view of a variable-resistance exercise machine with wireless communication for smart device control and interactive software applications 1600, according to an embodiment of the invention. According to the embodiment, an exercise machine 1600 may have a stable base 1601 to provide a platform for a user to safely stand or move about upon. Additional safety may be provided through the use of a plurality of integrally-formed or detachable side rails 1602, for example having safety rails on the left and right sides (with respect to a user's point of view) of exercise machine 1600 to provide a stable surface for a user to grasp as needed. Additionally, side rails 1602 may comprise a plurality of open regions 1605a-n formed to provide additional locations for a user to grasp or for the attachment of additional equipment such as a user's smart device (not shown) through the use of a mountable or clamping case or mount. Formed or removable supports 1606a-n may be used for additional grip or mounting locations, for example to affix a plurality of tethers (not shown) for use in interaction with software applications while a user is using exercise machine 1600.

Exercise machine 1600 may further comprise a rigid handlebar 1603 affixed or integrally-formed on one end of exercise machine 1600, for a user to hold onto while facing forward during use. Handlebar 1603 may further comprise a stand or mount 1604 for a user's smart device such as (for example) a smartphone or tablet computer, so they may safely support and stow the device during use while keeping it readily accessible for interaction (for example, to configure or interact with a software application they are using, or to select different applications, or to control media playback during use, or other various uses). Handlebar 1603 may be used to provide a stable handle for a user to hold onto during use for safety or stability, as well as providing a rigid point for the user to “push off” during use as needed, for example to begin using a moving treadmill surface. During use, a user may also face away from handlebar 1603, using exercise machine 1600 in the reverse without their view or range of motion being obscured or obstructed by handlebar 1603 (for example, for use with a virtual reality game that requires a wide degree of movement from the user's hands for interaction).

As illustrated, the base 1601 of exercise machine 1600 may be formed with a mild, symmetrical curvature, to better approximate the natural range of movement of a user's body during use. Common exercise machines such as treadmills generally employ a flat surface, which can be uncomfortably during prolonged or vigorous use, and may cause complications with multi-directional movement or interaction while a user's view is obscured, as with a headset. By incorporating a gradual curvature, a user's movements may feel more natural and require less reorientation or accommodation to become fluid and proficient, and stress to the body may be reduced.

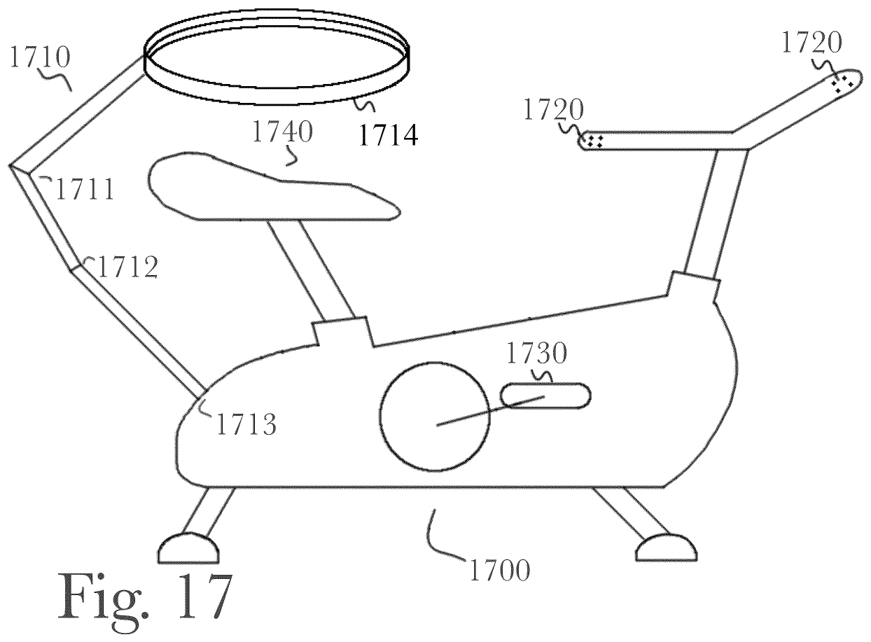

FIG. 17 is a diagram of an exemplary virtual reality or mixed reality enhanced exercise machine, illustrating the use of a stationary bicycle 1700 with hand controls on the handles 1720, and a belt-like harness attachment 1714. A stationary exercise bicycle device 1700, which may be of any particular design including a reclining, sitting, or even unicycle-like design, possesses two pedals 1730 as is common for stationary exercise bicycles of all designs. On handlebars of a stationary exercise bicycle may exist buttons and controls 1720 for interacting with a virtual reality or mixed reality augmented piece of software, allowing a user to press buttons in addition to or instead of pedaling, to interact with the software. A belt-like harness attachment 1714 is attached via a mechanical arm 1710 to a stationary exercise bicycle 1700, which may monitor motion and movements from a user during the execution of virtual reality software. A mechanical arm 1710 may have an outer shell composed of any material, the composition of which is not claimed, but must have hinges 1711, 1712, 1713 which allow for dynamic movement in any position a user may find themselves in, and angular sensors inside of the arm at the hinge-points 1711, 1712, 1713 for measuring the movement in the joints and therefore movement of the user. A stationary bicycle device 1700 may also have a pressure sensor in a seat 1740, the sensor itself being of no particularly novel design necessarily, to measure pressure from a user and placement of said pressure, to detect movements such as leaning or sitting lop-sided rather than sitting evenly on the seat.

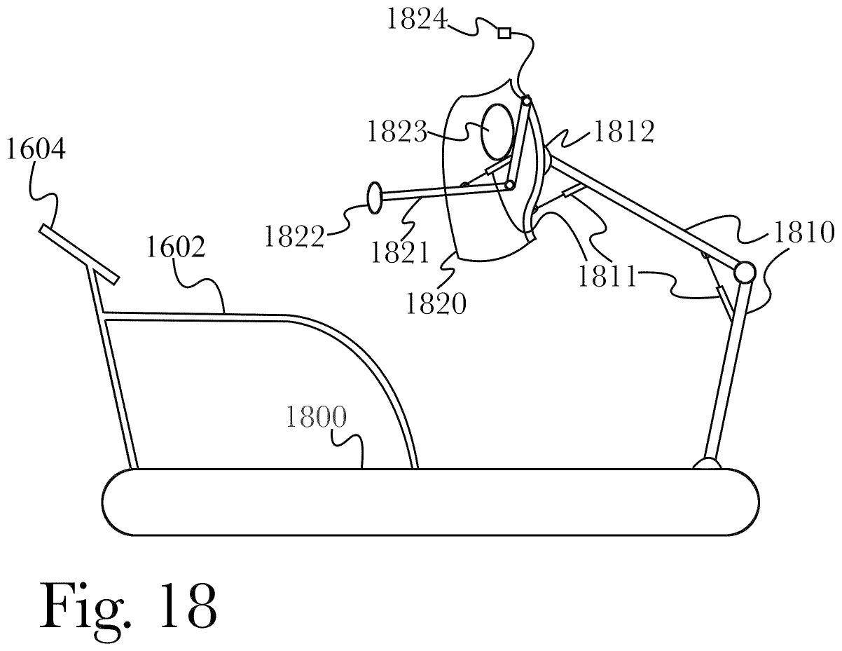

FIG. 18 is a diagram of another exemplary virtual reality or mixed reality enhanced exercise machine 1800, illustrating the use of a treadmill exercise machine 1000 a vest-type harness 1820 with a plurality of pistons 1811 to provide a hardware-based torso joystick with full-body tracking. According to this embodiment, a treadmill or other exercise machine 1000 may comprise a plurality of rigid side rails 1602 for a user to grip for support as needed during use (for example, as a balance aid or to assist getting on the machine and setting up other equipment properly) as well as a rigid stand or mount 1604 for a user's smartphone or other computing device, that may be used to operate a virtual reality or mixed reality software application. Exercise machine 1000 may further comprise a jointed arm 1810 or similar assembly that may be integrally-formed or removably affixed to or installed upon exercise machine 1000. Arm 1810 may utilize a plurality of pistons 1811 to provide for movement during use in order to follow the movements of a user's body, as well as to provide tension or resistance to motion when appropriate (for example, to resist a user's movements or to provide feedback) and motion detection of a user's movement during use by measuring movement of a piston 1811 or arm 1810 and optionally applying tension or resistance to piston 1811 to retard movement of arm 1810 and constrain user movement or simulate specific forms of physical feedback. For example, if a user is moving an avatar in a virtual reality software application, when the avatar encounters an obstacle such as another avatar, object, or part of the environment, resistance may be applied to piston 1811 to prevent the user from moving further, so that their avatar is effectively prevented from moving through the obstacle and thereby facilitating the immersive experience of a solid object in a virtual environment. Additional arms may be used for a user's limbs 1821 and may incorporate straps 1822 to be affix about a user's arm, wrist, or other body part (for example, when placed through an appropriate arm or limb hole 1823 in vest harness 1820 while worn), to incorporate more detailed movement tracking of a user's arms and/or legs rather than just torso-based tracking. A vest-type harness 1820 may be affixed to jointed arm 1810 using a movable joint 1812 such as a ball joint (for example) and used in place of a belt to allow for more natural movement or to provide greater area upon which to affix additional arms 1821, pistons 1811, or any of a variety of sensors, for example such as accelerometers or gyroscopes for detecting body orientation (not all optional sensors are shown for the sake of clarity). For example, a vest 1820 may have integrated feedback actuators for use in first-person software applications to simulate impacts or recoil, or it may incorporate heating or cooling elements to simulate different virtual environments while worn. Additionally, vest 1820 may incorporate electrical connectors 1824 for various peripheral devices such as additional sensors, tracking devices, controllers, or a headset, reducing the risk of tangles or injury by keeping cables short and close to the user so they cannot cause issues during movement or exercise.

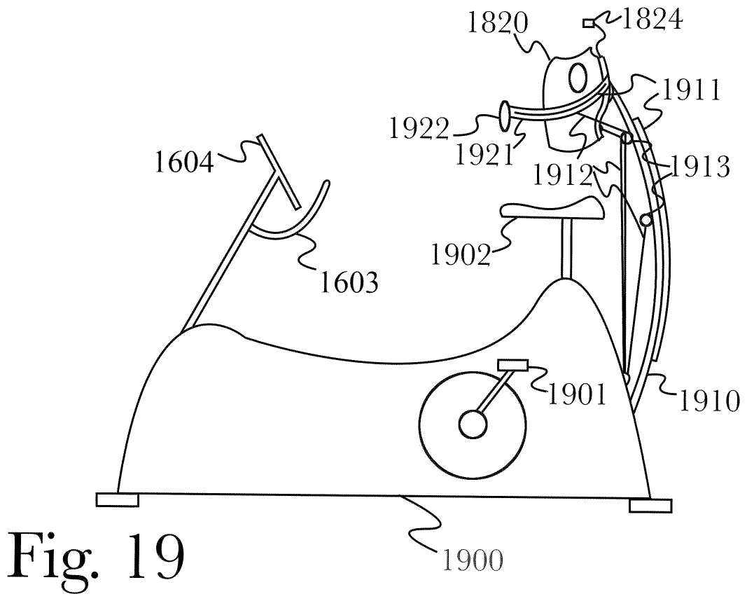

FIG. 19 is a diagram of another exemplary virtual reality or mixed reality enhanced exercise machine, illustrating the use of a stationary bicycle 1900 with a vest-type harness 1820 with a plurality of strain sensors 1911 and tethers 1912, according to an aspect of the invention. According to this embodiment, rather than a jointed arm 1810 and pistons 1811, a solid flexible arm 1910 may be used to detect user movement while positioned on a seat 1902 to use exercise machine 1000, for example while the user is seated to use pedals 1901 on a stationary bike or elliptical training machine. Through a plurality of strain gauges 1911 that detect the flexion or extension of the solid arm. Tethers 1912 may be used for either movement tracking or providing feedback to a user, or both, and may optionally be connected or routed through joints or interconnects 1913 to allow for a greater variety of attachment options as well more precise feedback (for example, by enabling multiple angles from which a tether 1912 may apply force, to precisely simulate different effects). Tethers may be operated by means of actuators such as motors, solenoids, pistons, or any other means of placing or releasing tension on the tether. For example, an end of the tether may be attached to a lever arm operated by a piston-type actuator, or may be attached to a reel operated by a motor-type actuator. Additional arms may be used for a user's limbs 1921 and may incorporate straps 1922 to be affix about a user's arm, wrist, or other body part, to incorporate more detailed movement tracking of a user's arms and/or legs rather than just torso-based tracking. Additional arms 1921 may also incorporate additional tethers 1912 and strain sensors 1911 to track movement and apply feedback to specific body parts during use, further increasing precision and user immersion. A vest-type harness 1820 may be used in place of a belt 1714 to allow for more natural movement or to provide greater area upon which to affix additional arms 1921, tether 1912, or any of a variety of sensors, for example such as accelerometers or gyroscopes for detecting body orientation (not all optional sensors are shown for the sake of clarity). For example, a vest 1820 may have integrated feedback actuators for use in first-person software applications to simulate impacts or recoil, or it may incorporate heating or cooling elements to simulate different virtual environments while worn. Additionally, vest 1820 may incorporate electrical connectors 1914 for various peripheral devices such as additional sensors, tracking devices, controllers, or a headset, reducing the risk of tangles or injury by keeping cables short and close to the user so they cannot cause issues during movement or exercise.

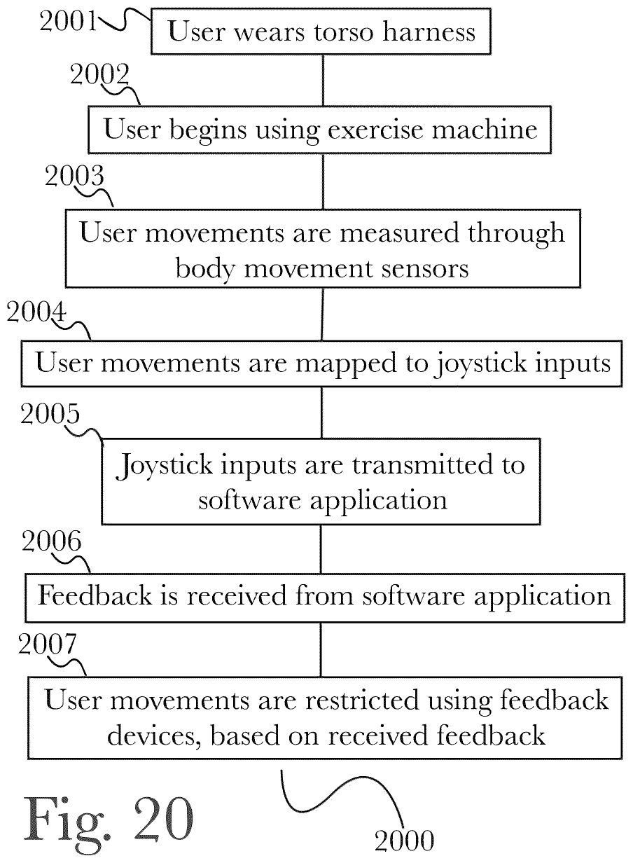

FIG. 20 is a flow diagram illustrating an exemplary method 2000 for operating a virtual and

mixed-reality enhanced exercise machine 1000, according to one aspect. According to the aspect, a user may wear 2001 a torso harness such as a belt 1714 or vest 1820 harness, while they engage in the use 2002 of an exercise machine. While using the exercise machine 1000, the user's movements may be detected and measured 2003 through the use of a plurality of body movement sensors such as (for example, including but not limited to) strain sensors 1911, tethers 1912, pistons 1811, or optical sensors 1201a-n. These measured user movements may then be mapped by the exercise machine 1000 operating as a control device 801 to correspond to a plurality of movement inputs of a virtual joystick device 2004. These virtual joystick inputs may then be transmitted 2005 to a software application, for example a virtual reality or mixed reality application operating on a user device such as (for example, including but not limited to) a smartphone 1030, personal computing device, or headset. The exercise machine 1000 may then receive feedback from the software application 2006, and may direct the operation of a plurality of feedback devices such as tethers 1912 or pistons 1811 to resist or direct the user's movement 2007 to provide physical feedback to the user based on the received software feedback.

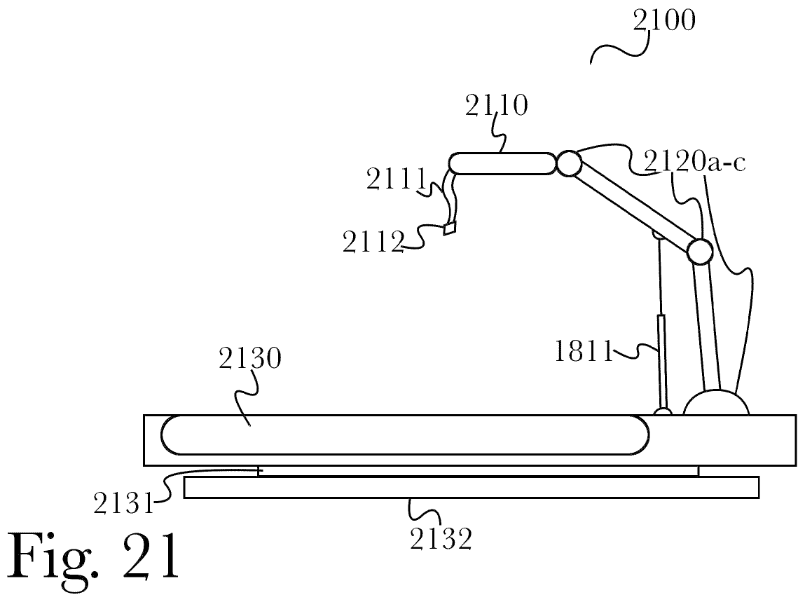

FIG. 21 is a diagram of another exemplary virtual reality or mixed reality enhanced exercise machine 2100, illustrating the use of a rotating platform, a waist belt 2110 and joints 2120a-c providing full range of motion. A virtual reality or mixed reality exercise machine 2100 may comprise an exercise machine such as (for example) a treadmill 2130 that has a movable or pivotable base 2132, for example via a turnstile 2131 or similar joint for allowing movement of the entire exercise machine 2100 while base 2132 remains stationary or fixed to the floor. A waist belt 2110 such as a padded hip belt or a support belt (such as those used for weight lifting or other physical activities, or those used for physical therapy or other medical uses) may be utilized with belt straps 2111 and buckle 2112 to fasten about a user's waist. Waist belt 2110 may be attached to exercise machine 2100 using a plurality of ball or similar joints 2120a-c configured to allow for full range of motion, enabling complex user movements and posture and allowing the use of full freedom of movement when interacting with software applications. A plurality of pistons 1811 or other means may be used, as described previously, to restrict or direct user movement or to provide feedback, enabling a variety of interaction and feedback options as well as providing a means to control and manipulate user movement both for immersion in virtual or mixed reality applications as well as for a variety of medical or therapeutic uses, such as preventing users from exceeding recommended range of motion during physical therapy or preventing movement past safety parameters.

### Exemplary Computing Environment

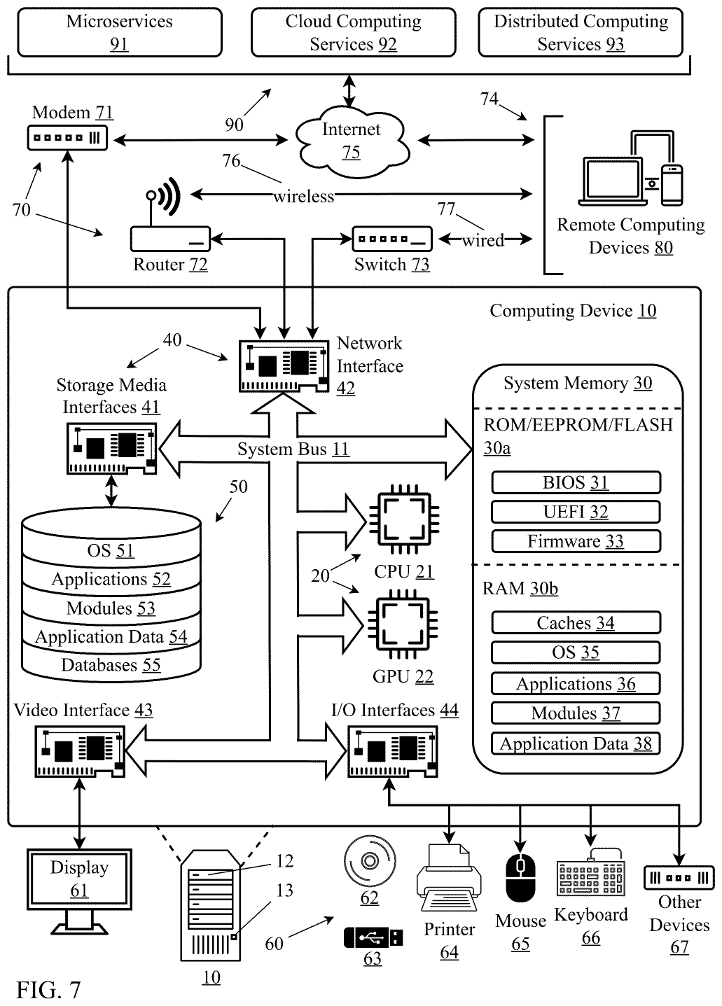

FIG. 7 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud-based services 90.

System bus 11 couples the various system components, coordinating operation of and data transmission between, those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 can be physically co-located or integrated into a single physical component, such as on a single chip. In such a case, some or all of system bus 11 can be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which can be used to store the desired content and which can be accessed by the computing device 10. Computing device may further comprise externally-accessible data ports or connections 12 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as “flash drives” or “thumb drives”) 63, printers 64, pointers and manipulators such as mice 65, keyboards 66, and other devices such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). However, the term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable or independently or semi-independently processing programming instructions. Further, computing device 10 may comprise one or more specialized processors such as a graphics processing unit (GPU) 22 configured to accelerate processing of computer graphics and images via a large array of specialized processing cores arranged in parallel.

System memory 30 is processor-accessible data storage in the form of volatile and/or nonvolatile memory. System memory 30 may be either or both of two types: non-volatile memory 30a such as read only memory (ROM), electronically-erasable programmable memory (EEPROM), or rewritable solid state memory (commonly known as “flash memory”). Non-volatile memory 30a is not erased when power to the memory is removed. Non-volatile memory 30a is typically used for long-term storage a basic input/output system (BIOS) 31, containing the basic instructions, typically loaded during computer startup, for transfer of information between components within computing device, unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b such as random access memory (RAM) is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non-volatile data storage device 50. Network interface 42 provides the necessary hardware interface for computing device 10 to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44.

Non-volatile data storage devices 50 are typically used for long-term storage provide long-term storage of data. Data on non-volatile data storage devices 50 is not erased when power to the non-volatile data storage devices 50 is removed. Non-volatile data storage devices 50 may be implemented using technology for non-volatile storage of content such as CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 50 may be non-removable from computing 10 as in the case of internal hard drives, removable from computing device 10 as in the case of external USB hard drives, or a combination thereof, but computing device will comprise one or more internal, non-removable hard drives using either magnetic disc or solid state memory technology. Non-volatile data storage devices 50 may store any type of data including, but not limited to, an operating system 51 for providing low-level and mid-level functionality of computing device 10, applications for providing high-level functionality of computing device 10, program modules 53 such as containerized programs or applications, or other modular content or modular programming, application data 54, and databases 55 such as relational databases, non-relational databases, and graph databases.

Applications (also known as computer software or software applications) are sets of programming instructions designed to perform specific tasks or provide specific functionality on a computer or other computing devices. Applications are typically written in high-level programming languages such as C++, Java, and Python, which are then either interpreted at runtime or compiled into low-level, binary, processor-executable instructions operable on processors 20. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between

computing device and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network. Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers may be employed. For example, secure socket layer (SSL) acceleration cards can be used to offload SSL encryption computations, and transmission control protocol/internet protocol (TCP/IP) offload hardware and/or packet classifiers on network interfaces 42 may be installed and used at server devices.

In a networked environment, certain components of computing device 10 may be fully or partially implemented on remote computing devices 80 or cloud-based services. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 30 for use) such processes and components may reside or be processed at various times in different components of computing device 10, remote computing devices 80, and/or cloud-based services 90.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, main frame computers, network nodes, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are microservices 91, cloud computing services 92, and distributed computing services.

Microservices 91 are collections of small, loosely coupled, and independently deployable computing services. Each microservice represents a specific business functionality and runs as a separate process or container. Microservices promote the decomposition of complex applications into smaller, manageable services that can be developed, deployed, and scaled independently. These services communicate with each other through well-defined APIs (Application Programming Interfaces), typically using lightweight protocols like HTTP or message queues. Microservices 91 can be combined to perform more complex processing tasks.

Cloud computing services 92 are delivery of computing resources and services over the Internet 75 from a remote location. Cloud computing services 92 provide additional computer hardware and storage on as-needed or subscription basis. For example, cloud computing services 92 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over the Internet on a subscription basis.

Distributed computing services 93 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power. These services enable parallel processing, fault tolerance, and scalability by distributing tasks across multiple nodes.

Although described above as a physical device, computing device 10 can be a virtual computing device, in which case the functionality of the physical components herein described, such as processors 20, system memory 30, network interfaces 40, and other like components can be provided by computer-executable instructions. Such computer-executable instructions can execute on a single physical computing device, or can be distributed across multiple physical computing devices, including being distributed across multiple physical computing devices in a dynamic manner such that the specific, physical computing devices hosting such computer-executable instructions can dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 10 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces such as an application program interface (API).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for controlling an application with a fitness device through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A fitness device gaming controller, comprising:
an exercise machine configured to:
receive user input from a manually operated control, or an observed hardware sensor value, or both;
create a dataset comprising at least a portion of the received user input;
transform the dataset to conform to an expected gaming controller input scheme; and
transmit the transformed dataset to a gaming device.

2. The system of claim 1, wherein the exercise machine is a treadmill device.

3. The system of claim 1, wherein the exercise machine is a stationary bicycle.

4. The system of claim 1, wherein the exercise machine is an elliptical training device.

5. The system of claim 1, wherein the gaming device is a gaming console.

6. The system of claim 1, wherein the gaming device is a personal computing device.

7. The system of claim 1, wherein the gaming device is a cloud-based gaming service.

8. The system of claim 1, wherein the exercise machine further comprises a plurality of electronic connectors to accept external devices.

9. The system of claim 8, wherein the exercise machine is further configured to receive additional input from a connected external device, and the dataset comprises at least a portion of the additional input.

10. The system of claim 9, wherein the external device is a biometric sensor.

11. The system of claim 9, wherein the external device is a personal computing device.

12. The system of claim 11, wherein the personal computing device is a smartphone.

13. The system of claim 11, wherein the personal computing device is a smartwatch.

14. The system of claim 11, wherein the personal computing device is a computer built in to and integrated with the exercise machine.
